(19) United States
(12) Patent Application Publication
(10) Pub. No.: US 2019/0374552 A1
(43) Pub. Date: Dec. 12, 2019

(54) MULTIFUNCTIONAL FORMULATIONS AND METHODS TO CONTROL DERMATITIS AND PRURITUS

(71) Applicant: George Edward Hoag, Storrs, CT (US)

(72) Inventor: George Edward Hoag, Storrs, CT (US)

(21) Appl. No.: 16/346,450

(22) PCT Filed: Feb. 11, 2017
(86) PCT No.: PCT/US2017/059712

§ 371 Date: Apr. 30, 2019

Related U.S. Application Data
(60) Provisional application No. 62/416,482, filed on Nov. 2, 2016.

(51) Int. Cl. A61K 31/573; A61K 31/352; A61K 36/55; A61K 36/736; A61K 47/26; A61K 47/46; A61K 31/593; A61K 31/4174

(52) U.S. Cl. CPC A61K 31/573; A61K 31/352; A61K 36/55; A61K 36/736; A61K 45/06; A61K 47/46; A61K 31/593; A61K 31/4174; A61K 47/26

(57) ABSTRACT

This disclosure relates to multifunctional formulations, methods of manufacture and methods of use of natural topical and oral compositions to treat various dermatitis and pruritus conditions in mammals. In particular, this disclosure relates to a multifunctional topical pharmaceutical composition effective to treat dermatitis and associated pruritus comprising at least one natural plant extract TRPV1 antagonist, at least one natural plant extract is a TRPA1 antagonist, and a carrier. Also, in particular, this disclosure also relates to a multifunctional method to treat histamine induced and non-histamine dermatitis and associated pruritus in mammals from one or more of non-atopic and atopic dermatitis (AD), contact dermatitis, allergenic contact dermatitis (ACD), psoriasis, eczema, infestations, urticarial, nociceptive, neuropathic, neurogenic, psychogenic pruritus comprising treating with a composition comprising at least one natural plant extract TRPV1 antagonist, at least one natural plant extract is a TRPA1 antagonist, and a carrier.

### BACKGROUND OF THE DISCLOSURE

### 1. Field of the Disclosure

This disclosure relates to multifunctional formulations, methods of manufacture and methods of use of natural topical and oral compositions to treat various dermatitis and pruritus conditions in mammals. More particularly, this disclosure relates to the use of compositions comprised of natural plant extract compounds that are multifunctional TRPA1 and TRPV1 ion channel antagonists, CGRP antagonists, Cannabinoid CB2 antagonists, MrgprA3-coupled protein receptor antagonists, TRPM-8 agonists and COX-2 inhibitors.

### 2. Description of the Related Art

Itching, technically known as pruritus, is experienced by many mammals, including humans. Partials in humans can be caused by various underlying diseases, including those with dermatological, neurological and systemic disorders and also from drugs with pruritic side effects. One group of the skin's sensory nerves has histamine receptors that can be blocked by antihistamines to help stop itch. However, activation of the skin's sensory nerves by known skin irritant compounds; inflammation, from asthma and allergies to skin rash; eczema and psoriasis create histamine-independent itch that is not currently treatable most drugs. Treatments for histamine-independent pruritus are ineffective when antihistamine products, such as diphenhydramine are used. While scratching usually provides temporary itch relief, scratching is believed to prolong and to be involved in itch becoming chronic by secondary mechanisms such as drying of the skin, inflammation, and infection. This phenomena is frequently referred to as the itch-scratch-itch cycle.

There are a variety of methods to categorize itch. Pruriceptive itch is peripherally induced pruritus arising from skin diseases such as dry skin, atopic dermatitis (AD), contact dermatitis, psoriasis, infestations (e.g. scabies, pediculosis), and urticaria. Swelling of the deeper layers of the skin, known as angioedema, often occurs with hives. Hives often severely itch and can occur on any part of the skin.

Non-histaminic pruritus is associated with dermal disorders including alopecia areata, asteatotic dermatitis, atopic dermatitis, cutaneous B-cell lymphoma, cutaneous T-cell lymphoma, dermatitis herpetiformis, discoid eczema, hand eczema, ichthyosis, idiopathic dermatitis, mycosis fungoides, non-atopic dermatitis, notalgia paresthetica, psoriasis, prurigo nodularis, seborrheic dermatitis, Sezary syndrome and varicose eczema.

Neurogenic itch is centrally induced pruritus caused by systemic disorders such as chronic liver disease, (cholestasis), chronic renal failure, and thyroid dysfunction. Neuropathic itch is pruritus due to a primary neurological disorder of the central or peripheral nervous systems, and can be induced by certain brain tumors, multiple sclerosis, peripheral neuropathy (e.g. postherpetic (herpes-induced pruritus), nerve compression or irritation (e.g. notalgia paresthetica, brachioradial pruritus). Psychogenic itch is pruritus related to psychological or psychiatric disorders such as parasitophobia and obsessive-compulsive disorder, leading to neurotic excoriations.

Pruritus has been found to be the most frequent complaint (64%) among patients suffering from psoriasis. Psoriatic pruritus does not respond to antihistaminic drugs and is a non-histaminic symptom of psoriasis. Eczema is known as the “itch that rashes” and similar to psoriasis, does not respond to antihistaminic drugs, and 87%-100% of patients with eczema have chronic itch.

Methods to reduce human suffering from non-histaminergic pruritus typically utilize medications, such as corticosteroids, opioid receptor antagonists, antiepileptic drugs, or local anesthetics to relive itch. Unfortunately, these medications treat symptoms and do not directly treat the cause of the pruritus.

Effective treatments are needed for non-histaminergic pruritus that are effective and free from debilitating side effects.

Chronic itch associated with skin and systemic diseases is insensitive to antihistamine treatment, and even allergic itch is only marginally inhibited by histamine receptor antagonists. However, the mechanisms underlying histamine-independent itch is very much a field of emerging science.

Pruritus in dogs is also common and usually caused by parasites, various allergens or underlying diseases. Canine pruritus is rarely successfully treated with antihistamines and dogs with this condition almost exclusively suffer from non-histaminergic itch. Pruritus in cats is usually caused by parasites or allergens or other conditions and react to itchiness in similar to dogs with scratching, licking and biting. Up to 50 percent of cats, however, have histamine-sensitive types of pruritus, while the remaining pruritic cats suffer from non-histaminergic forms of pruritus.

Pruritus in horses is extremely common and is one of the most common reasons for horse owners seek veterinarian consultation. A horse with itchy skin will rub up against fences, stalls, trees, or other objects attempting to scratch the itch and may bite or lick its skin to the point of causing bleeding or damage to the skin. The most common causes of allergic itching in horses are insect bites, food allergies including allergens in horse feed, and itching due to seasonal allergens. Pruritus in horses is seldom treated successfully with antihistamines, indicating that pruritus in horses usually is non-histaminergic pruritus.

Medical researchers involved in the field of itch have proposed a novel pruriceptive system, within which itch-inducing peripheral mediators (pruritogens), itch-selective receptors (pruriceptors), sensory afferents and spinal cord neurons, and defined, itch-processing central nervous system regions display complex, layered responses to itch. Several cell types contribute to chronic itch pathologies and sensations. Tissue-resident cells in the skin, such as keratinocytes and cells that infiltrate during inflammation, such as lymphocytes, mast cells, and eosinophils, release pruritogens and activate primary afferent neurons.

The skin is highly innervated and contains a dense network of sensory afferents. Among the unmyelinated C-afferent axons (also referred to as C-nociceptors), approximately 80% are mechano-sensitive polymodal nociceptors, which respond to mechanical, thermal (heat), and chemical stimuli and approximately 20% do not respond to mechanical stimulation but are activated by chemical stimuli.

Pruritogens are detected by the free nerve endings of primary afferent C-fibers located within the epidermis. Itch is mediated by both histamine-sensitive and histamine-insensitive C-fibers, although most forms of chronic itch are insensitive to antihistamine treatment and are mediated by histamine-insensitive neurons.

Skin-localized sensory afferents are involved in the neuronal processing of multiple sensory modalities (e.g. pain, itch, touch, thermosensation). Recent research suggests that transient receptor potential (TRP) ion channels not only act as polymodal cellular sensors on sensory neurons but are also functionally expressed by many non-neuronal cell types. This is especially true in the skin, one of the largest organs of the body, where they appear to be critically involved in regulating various cutaneous functions both under physiological and pathophysiological conditions.

In vertebrates, the somatosensory system can discriminate small changes in ambient temperature, which activate nerve endings of primary afferent fibers. These thermosensitive nerves are further distinguished into those that detect either innocuous (non-painful) or noxious (painful) temperatures. The mechanism for the perception of cooling sensation of the skin can be ascertained with as little as a 1° C. change in temperature. When the temperature on the skin decreases and approaches 15° C., however, the perception of cold pain is sensed via nociceptors.

Thermosensitive afferent nerve fibers that carry impulses from the body to the brain express ion channels of the transient receptor potential (TRP) family and respond at distinct temperature thresholds. This comprises the molecular basis for thermosensation. Transient Receptor Potential channels are a group of ion channels found in cells of many animal cell types. Many of these channels mediate a variety of sensations like the sensations of pain, hotness, warmth or coldness. The main temperature sensors belong to the TRP cation channel family, but the actual mechanisms underlying the marked temperature sensitivity of opening and closing (gating) of these channels is still largely unknown. While activation of various TRP ion channels causes stimuli such as that the result of chemical, mechanical or thermally induced pain, these same TRP ion channels can be inhibited to decrease or eliminate the sensing of pain and pruritus.

TRP channels represent a heterogeneous system oriented towards environment perception, and participating in sensing visual, gustatory, olfactive, auditive, mechanical, thermal, osmotic and pruritogenic stimuli. The TRP family of channels, currently contains more than 50 different channels and 27 in humans. TRP channel gating is operated by both the direct action on the channel by a plethora of exogenous and endogenous physicochemical stimuli. A large amount of evidence shows that TRPA1 ion channel plays a key role in the detection of pungent or irritant compounds, including compounds contained in different spicy foods, such as allyl isothiocyanate (in mustard oil), horseradish, allicin and diallyldisulfide in garlic, cinnamaldehyde in cinnamon, gingerol (in ginger), eugenol (in cloves), methyl salicylate (in wintergreen), menthol (in peppermint), carvacrol (in oregano), thymol (in thyme and oregano). In addition, environmental irritants and industry pollutants, such as acetaldehyde, formalin, hydrogen peroxide, hypochlorite, isocyanates, ozone, carbon dioxide, ultraviolet light, and acrolein (a highly reactive α,β-unsaturated aldehyde present in tear gas, cigarette smoke, smoke from burning vegetation, and vehicle exhaust), have been recognized as TRPA1 activators.

Recent research by Wilson et al. (2011) on mice indicates that Transient Receptor Potential Ankyrin 1 (TRPA1) is essential for acute itch. It has been demonstrated that TRPA1 may contribute to acute endogenous histamine independent itch sensations. In addition to pain and itch, sensory nerves promote acute and delayed inflammatory responses in the skin in Allergenic Contact Dermatitis (ACD) and other pathological skin conditions.

TRPA1 is a TRP channel that functions as a receptor for noxious cold temperatures and various tissue and skin irritations, for example those caused by parabens, urushiol, and burns from alkaline compounds, as well as cooling compounds, such as menthol, as well as methyl salicylate. Several of the TRPA1 activators are also known as triggers of migraine attack, and pruritus. Because TRAP is an excitatory ion channel targeted by cold nociception, itch and inflammatory pain, TRPA1 is a promising target for use in identifying analgesic drugs that could inhibit TRPA1.

Recent research by Liu et al. (2013) on mice exhibiting antihistamine-resistant scratching behavior ACD following exposure to the two haptens; oxazolone and urushiol, showed pharmacological inhibition of scratching when exposed to a synthetic TRPA1 antagonist HC-030031. Additionally mice with genetically ablated TRPA1 showed resistance to antihistamine-resistant scratching behavior ACD following exposure to haptens, oxazolone and urushiol. Liu et al. (2013) concluded that TRPA1 is a crucial component of the inflammatory response in contact dermatitis following the cutaneous hapten oxazolone challenge. They also concluded that constant TRPA1-dependent activation of and feedback from sensory neurons is necessary to maintain chronic dermatitis and establish chronic pruritus, with Substance P (SP) playing a key role in histamine (and mast cell)-independent inflammation and pruritus. Toth et al. (2014) concluded that TRPA1 exerts a pro-inflammatory role in the skin, most probably via an orchestrated interplay between neurogenic and non-neurogenic mechanisms. TRPA1 is a downstream transduction channel onto which multiple histamine-independent itch pathways converge. Naturally occurring TRPA1 agonists include: allyl-isothiocyanate (wasabi, mustard oil); benzyl-isothiocyanate (yellow mustard); phenylethyl-isothiocyanate (brussels sprouts); isopropyl-isothiocyanate (Nasturtium seeds); methyl-isothiocyanate (Capparis spinose); cinnamaldehyde (Cinnamomum cassia, Cinnamomum zeylanicum); eugenol (clove oil); gingerol (Zingiber officinale); methyl salicylate (wintergreen oil); allicin (Allium Sativum); carvacrol (Origanum genus); menthol (Mentha piperita 1, Mentha arvensis).

Cross-talk of TRPs and many neuropeptides is also emerging and an important area of research. While activation of various TRP ion channels causes stimulation of these nociceptors, resulting in chemical, mechanical or thermally induced pain, these same TRP ion channels can be inhibited to decrease or eliminate the sensing of pain and pruritus.

Various itch mediators and receptors have been identified in the skin. The Mas-related G protein-coupled receptor A3 (MrgprA3) has been identified by Liu et al. (2009) as a receptor for non-histaminergic itch. This work was expanded upon by Han et al (2013) who identified an itch-specific population of MrgprA3-expressing neurons in the dorsal root ganglion (DRG). These neurons exclusively supply neurons to the epidermis, respond to multiple pruritogens, and form synapses with gastrin-releasing peptide receptor-positive (GRPR⁺) neurons in the dorsal horn of the spinal cord. When these neurons are ablated scratching is reduced in response to prutitogen injection.

Cross-talk between keratinocytes, immune cells and neighboring neuronal afferent endings through the release of secreted compounds (for example, the result of neurogenic inflammation factors), further modulates cell responses and itch pathway output. Subsets of sensory neuron afferents innervate the skin and mediate itch signaling. The cytokine thymic stromal lymphopoietin (TSLP), an IL-7-like cytokine, activates neurons via a TSLP receptor, which leads to the opening of TRPA1 channels. The activation of TRPA1 leads to neuronal depolarization, action potential firing and the transmission of itch signals from the periphery to the CNS.

It has been shown that inflammation induced by carrageenan involves cell migration, plasma exudation, and production of mediators, such as nitric oxide, prostaglandin E₂, interleukin (IL)-i p, IL-6, and tumor necrosis factor (TNF-a). Borneol has been shown to reduce leukocyte migration induced by injection of the inflammatory agent carrageenan. Therefore, borneol may inhibit interleukin (IL)-i p, IL-6, and tumor necrosis factor (TNF-a). Additional studies have demonstrated that borneol inhibits TNF-a and IL-6. The release of IL-I β, IL-4, IL-5 and TNF-a by stimulated lymphocytes has been decreased by 1,8-cineole.

Pruritic responses are triggered by somatosensory neurons, with several itch-inducing agents acting through a pathway involving the ion-channel TRPV1. TRPV1 was the first to be described member of the TRPV subfamily. It was identified in rat DRGs as the receptor for capsaicin, the pungent ingredient of red hot chili peppers. Agonist induced silencing of TRPV 1-expressing neurons was shown to result in a profound loss of all itch responses. The role of TRPV1 in inflammatory and neuropathic states is well established. In addition to capsaicin being a TRPV1 agonist, TRPV1 is also activated by diverse stimuli including noxious temperatures (near 42° C.), extracellular acidic pH and bioactive lipids such as lysophosphatidic acid (LP A), herbal compounds, such as piperine, eugenol, urushiol, zingerone, resiniferatoxin, gingerol, zingerone, evodiamine, cannabidiol, polygodial, isovellera, camphor, vanillotoxin and seasonal allergens. Because TRPV1 plays a central role in the development of pruriceptive itch on both sensory neurons and non-neuronal cutaneous structures, targeting the TRPV1 to alleviate its activity, in addition to targeting TRPA1 is a promising therapeutic strategy to treat itch which is one composition of this instant disclosure.

Schwartz, et al (2013) studied the combination of TRPA1 and TRPV1 channels in pancreatic inflammation and pain-related behaviors in the transition of acute pancreatitis (AP) to chronic pancreatitis (CP) in mouse models. They reported treatment using a combination of TRPA1 and TRPV1 antagonists, when initiated prior to week 3 of AP bouts, decreased pancreatic inflammation and pain-related behaviors and also blocked development of histopathological changes in the pancreas and upregulation of TRPV1, TRPA1 and Phospho-ERK (pERK) (a nociceptor marker) in pancreatic afferents. Continued treatment with TRP antagonists blocked development of CP and pain behaviors even when mice were challenged with seven more weeks of twice per week caerulein, a pancreatitis chemical inducer. Schwartz, et al (2013) demonstrated that a multifunctional approach using synthetic TRPA1 and TRPV1 antagonists was efficacious in preventing the progression of AP to CP.

In a study conducted by Gualiani, et al (2015), these authors observed that two synthetic compounds ADM 09 and ADM 12, each behaved as dual TRPA1 and TRPV1 antagonists and blocked the TRPA1 agonist AITC, and the TRPV1 agonist capsaicin. These authors additionally reported that in mouse models ADM 12 was able to revert inflammatory trigeminal neuralgia, a chronic pain condition that affects the trigeminal nerve, which carries sensation from the face to the brain. Based on this study it appears that ADM 09 and ADM 12, synthetic compounds, exhibited multifunction TRPA1 and TRPV 1 antagonist capabilities.

Yet another recent research effort, by Perez-Faginas, et al (2016) reported that a high throughput screening campaign identified some β,γ-diamino ester derivatives as TRP modulators with the capabilities to block the agonist-induced calcium influx in cells expressing human TRPV1, TRPM8 and TRPA1 channels. They were able to develop antagonists for each channel, as well as dual TRPV1/TRPM8 and TRPM8/TRPA1 antagonist ligands. This research effort by Perez-Faginas, et al (2016) clearly points out the efficacy of the strategy of multifunctional TRP antagonists.

Psoriasis is an inflammatory skin disease with an incompletely understood causation. It is characterized by red and scaly skin lesions formed by a high rate of epidermal keratinocyte proliferation driven by cytokines secreted by activated resident immune cells, an infiltrate of T cells, dendritic cells and cells of the innate immune system, as well as the keratinocytes themselves. Prescription cytokine drugs are currently available to treat psoriasis that target tumor necrosis factor (TNF)-α, interleukin (IL)-12/23, IL-17, and IL-23. These drug are administered either by injection or intravenously. To date, there is only one prescription cytokine drug for the treatment of eczema that targets the cytokine IL-4/13.

Matta, J. A. (2007) reported that Omega-3 polyunsaturated fatty acids were novel targets for TRPV1. Specifically they reported that docosahexaenoic acid exhibits the greatest efficacy as a TRPV1 agonist. However, eicosapentaenoic acid and a-linolenic acid were markedly more effective inhibitors. Comparatively eicosapentaenoic acid but not docosahexaenoic acid profoundly reduced capsaicin-evoked pain-related behavior in mice. Very recently Morales-Lazaro, et al (2016) reported a naturally occurring monounsaturated fatty acid, oleic acid, an Omega-9 fatty acid, inhibited TRPV1 activity, and also pain and itch responses in mice by interacting with the vanilloid (capsaicin)-binding pocket and promoting the stabilization of a closed state conformation.

It is evident that the field of natural plant-based compounds, including borneol and 1,8-cineole, as well as plant and animal sources of Omega-3 fatty acids is leading to a recognition that these compounds have the ability to block cytokines involved in many diseases.

Hyaluronan (HA) is an anionic linear polymer that is expressed in the extracellular matrix (ECM) of mammalian tissues, where it forms loose and elastic matrices. In joints, HA is continuously secreted by the lining cells of the synovial membranes and provides a protective rheological buffer that reduces the force transmitted by joint movements to joint tissues, including nociceptive nerve endings.

Recently, Caires, R. et al, (2015) investigated the analgesic effects of HA in joints through the modulation of TRPV1 ion channel activity in nociceptive terminals. The pharmacological modulation of TRPV1 has been shown to produce anti -nociception in arthritis animal models. Caires, R. et al. (2015) observed that HA inhibits TRPV1 channel activity and reduces action potential firing in nociceptive neurons and that it shows a previously unknown molecular mechanism that explains the attenuation by HA of peripheral nociceptor activity and pain. These authors demonstrated that TRPV1 channels are molecular targets of HA and that in the presence of HA, TRPV1 opens less frequently, thereby decreasing the excitability of peripheral nociceptive neurons and reducing their responsiveness to noxious stimuli. Importantly, Caires, R. et al. (2015) observed that, HA selectively modulates TRPV1 ion channel function and that related TRP channels associated with sensory transduction of noxious and thermal stimuli such as TRPA1 and TRPM8, were not affected by HA.

Recently, Schlesinger et al. (2012) evaluated the efficacy and tolerability of hyaluronic acid sodium salt gel 0.2% in the treatment of facial seborrheic dermatitis. These authors concluded that the application of hyaluronic acid sodium salt gel 0.2% twice a day after facial cleansing improved the scale, erythema, pruritus, and PGA of subjects diagnosed with facial seborrheic dermatitis.

In recent research published in 2013, it was concluded that an effective analgesic compound would activate TRPM8 (i.e., desirable cooling, reducing pain and/or anti-inflammatory properties) and inhibit TRPA1 (i.e., undesirable causation of cold pain and inflammation) but not activate TRPV1 (i.e., undesirable causation of hot pain and inflammation). These researchers reported that 1,8-cineole (eucalyptol) activates human TRPM8 (hTRPM8) without activating hTRPA1. They also demonstrated that 1,8-cineole did not activate hTRPV1 or hTRPV2, 1,8-cineole is present in Eucalyptus oil from several species in highly varying concentrations (less than 5 percent to greater than 80 percent), in several Rosmarinus officinalis chemotypes (up to −50 percent) and in Salvia lavandulifolia (up to −25 percent). It has been shown that TRPM8 activation decreases inflammation and pain. While TRPM8 activation by menthol was reported by these researchers, it did not decrease human inflammatory response, likely because it also activated TRPA1, which causes inflammation. Further, application of menthol with 1,8-cineole significantly reduced irritation probably through inhibition of TRPA1 by 1,8-cineole. As a follow-up to this research published in 2013, an additional study was published by the same research group (Takaishi et al., 2014) on the role of several monoterpene analogs of camphor and their ability to inhibit hTRPA1. They reported that 1,8-cineole, camphor, borneol, 2-methylosoborneol, norcamphor and fenchyl alcohol did not activate hTRPA1 and that borneol, 2-methylosoborneol and fenchyl alcohol at 1 mM completely inhibited hTRPA1 activation by menthol and allyl isothiocyanate (mustard oil) at 1 mM and 10 uM, respectively. It was found that TRPA1 activation by 20 uM AITC was inactivated (IC-50 concentration) in order from lowest to highest concentration by 2-methylosoborneol (0.12 mM), borneol (0.20 mM), fenchyl alcohol 0.32 mM, camphor (1.26 mM) and 1,8-cineole (3.43 mM).

Natural and synthetic sources of borneol exist. Natural sources of borneol are preferred, including Thymus satureioides (Red Thyme Borneol Type Morrocco) and Cinnamomum burmanni (Mei Pian Tree). Additional sources of natural occurring borneol in plants exist and could be incorporated into compositions in this disclosure.

Recently, researchers have reported that in addition to its antinociceptive action and anti-inflammatory role, TRPM8, is a promising therapeutic target for treating internal inflammatory diseases such as colitis and inflammatory bowel disease.

Recently, Anderson (2017) in a small clinical trial (n=13) reported that thermal cold-stimuli inhibited histaminergic itch in a stimulus-intensity-dependent manner. A similar inhibitory tendency was observed on wheal reactions and neurogenic inflammation. They also applied chemical proxies of cold-stimuli, including 1-menthol, and reported significant inhibitory effects on histaminergic itch with an antipruritic effect-size comparable to that of 5% topical doxepin.

The plant genus Cannabis is a member of the plant family Cannabaceae, and there are 3 primary cannabis species which vary in their biochemical constituents: Cannabis sativa, Cannabis indica, and Cannabis ruderalis. In general, cannabis that has high levels of the psychoactive cannabinoid, delta9-tetrahydrocannabinol (A9-THC), and low levels of the non/antipsychoactive cannabinoid, cannabidiol (CBD), is referred to as “marijuana”. Cannabis that has high levels of CBD, and very low insignificant levels of A9-THC, is referred to as “industrial hemp,” or “hemp,” and has no psychoactive effects (Baron, et al., 2015). Recent advancements in selective breeding has enabled Cannabis sativa L. to yield high concentrations of CBD and low concentrations of THC. CBD concentrations in these hybrid Cannabis sativa L. plants enable CBD yields of 15% and higher and THC yields of 1% and lower.

A breakthrough in the understanding of how cannabis works in the brain occurred with the discovery of endogenous cannabinoids and receptors. The endocannabinoid system is widely distributed throughout the brain and spinal cord, and plays a role in many regulatory physiological processes including inflammation and nociception/pain. The endocannabinoid system consists of the cannabinoid 1 (CB1) and 2 (CB2) receptors, the endogenous cannabinoid receptor ligands (endogenous cannabinoids) n-arachidonoylethanolamine (anandamide, or AEA) and 2-arachidonoylglycerol (2-AG), as well as their degrading enzymes fatty acid amide hydrolase (FAAH) and monoacylglycerol lipase, respectively. Mechoulam (2013) reported that the CB1 receptor mediated anti-inflammatory effects of cannabinoids are suspected to be secondary to inhibition of arachidonic acid conversion by cyclooxygenase although CB2 receptor activation induces immunosuppression, which also reduces inflammation. Because of this, cannabinoid compounds including A9-THC and CBD have become of interest in pain and topical pain relief. This is particularly true of CBD, which exhibits no psychoactive effects.

Two types of dermatitis can also be generally classified as “atopic dermatitis” and “non-atopic dermatitis”. There are no apparent outwardly clinical signs that differentiate these diseases. Human patients with atopic dermatitis are mostly children and most often have immunoglobulin E (IgE)-mediated sensitivity to allergens. Humans with non-atopic dermatitis do not have IgE-mediated dermatitis and are mostly adults. Importantly, both pruritus associated with atopic dermatitis and pruritus associated with non-atopic dermatitis are virtually all non-histaminic forms of pruritus and do not substantially respond to antihistamine treatments.

Allergic contact dermatitis (ACD) is a common condition characterized by hypersensitivity to allergens, inflammation, and pruritogens in contact with the skin. Contact dermatitis can be caused by increased sensitivity and resulting epicutaneous irritation from environmental, nutritional or occupational allergens. Other contact allergens include metals, such as nickel and cobalt, fragrance constituents, detergents, and food chemicals.

A common form of contact dermatitis is the result of dermal exposure to urushiol, an oil produced by poison ivy, poison oak and poison sumac. About 85 percent of people are allergic to poison ivy, poison sumac or poison oak. Dermal exposure to urushiol is the most frequent cause of contact dermatitis in the United States. A major issue with ACD is persistent pruritus or itching, which leads to scratching and subsequently damages the skin and enables further access by allergens and pathogens. One of the major obstacles for the therapy of contact dermatitis, such as that caused by urushiol, is histamine-independent inflammation and pruritus.

In sensitized individuals, exposure to urushiol from poison ivy, oak or sumac induces severe inflammation, blistering, and pruritus or the skin, with symptoms lasting for up to weeks. The American Academy of Dermatology estimates that there are up to 50 million cases of urushiol-induced dermatitis annually in the United States alone, accounting for 10% of all lost-time injuries in the United States Forest Service.

Urushiol causes an eczematous contact dermatitis characterized by redness, swelling, papules, vesicles, blisters, and streaking. People vary greatly in their sensitivity to urushiol. Urushiol does not trigger an immune system response in approximately 15 percent to 30 percent of people. At least 25 percent of people have a very strong immune response resulting in severe symptoms. Since the skin reaction is an allergic one, people may develop progressively stronger reactions after repeated exposures, or have no immune response on their first exposure but show sensitivity on subsequent exposures. Approximately 80% to 90% of adults will get a rash if they are exposed to 50 micrograms of purified urushiol. Some people are so sensitive that it only takes a trace of urushiol (two micrograms) on the skin to initiate an allergic reaction.

Treatment of urushiol dermatitis and pruritus consists of two phases: stopping the urushiol contact with the skin that is causing the reaction, which must be done within minutes of exposure and subsequently, reducing the pain and/or itching.

Primary treatment involves washing exposed skin thoroughly with soap, water, and friction as soon as possible after exposure is discovered. Soap or detergent is necessary because urushiol is an oil; friction, with a washcloth or something similar, is necessary because urushiol adheres strongly to the skin. Commercial removal preparations, which are available in areas where poison ivy grows, usually contain surfactants, such as the nonionic surfactant Triton X-100, to solubilize urushiol, while some products also contain abrasives.

Some of the more common treatments for urushiol ACD dermatitis and associated pruritus include hydrocortisone creams and ointments, diphenhydramine gels, calamine lotion and a proprietary product, Ivy Dry®. Hydrocortisone, diphenhydramine, and calamine lotion to some extent reduce pain and itching and offer only temporary relief from the itching associated with urushiol induced contact dermatitis, and they do nothing to remove urushiol from the skin. Likewise, Ivy Dry® provides essentially a cooling effect that is only temporary, and may, if used before the urushiol binds to the skin, remove some of the urushiol. Similarly, an unpatented product sold under the mark Tech-Nu® provides some relief for poison ivy exposure. The main active ingredient in Tech-Nu is octylphenoxy-polyethoxyethanol, also known as the non-ionic surfactant Triton X-100. The four octyl groups of this chemical are too large to fully emulsify the non-polar moieties in urushiol. Thus, the action of Tech-Nu renders the urushiol only partially inactive. Since the urushiol remains partially active and continues to cause irritation, only temporary relief is provided and multiple applications are necessary.

U.S. Pat. No. 7,008,963 discloses a method for treating urushiol induced contact dermatitis, comprising the steps of: providing an aqueous topical composition comprising nonoxynol-9; sodium lauroyl sarcosinate (SLS); and water; applying the composition to an affected area; permitting the composition to remain on the affected area a sufficient amount of time to enable the composition to cause an effect; and removing the composition from the affected area. This patent also discloses methods that use of acetylated lanolin alcohol, providing polyethylene granules or pumice, and EDTA. In this patent is it also disclosed that the addition of d-limonene can be used in combination with an ethoxylate to create an effective urushiol binding or neutralizing agent although the inventor claims that d-limonene is somewhat less effective, substitute for SLS. A limitation of this patent is that it is focused on the removal of urushiol following exposure to minimize the subsequent dermatitis and pruritus, but it does not mechanistically reduce dermatitis and pruritus once these symptoms have appeared.

U.S. Patent Application Publication US 20060177400 A1 discloses a topical formulation for the treatment of urushiol induced contact dermatitis comprising two or more micelle-forming surfactants, wherein micelle formation is generally enhanced and the formation of mixed micelles occurs. The formation of mixed micelles when an anionic surfactant or an amphoteric surfactant (a first medicament) is combined with a nonionic polyoxyethylene-based surfactant (a second medicament) is suggested for the reduction in skin irritation and increased effectiveness for removal of oils such as urushiol from the skin. A limitation of this patent application is that it is focused on the removal of urushiol following exposure to minimize the subsequent dermatitis and pruritus, but it does not mechanistically reduce dermatitis and pruritus once these symptoms have appeared.

U.S. Pat. No. 7,858,570A discloses method for treating a condition caused by the contact of skin with a source of urushiol, the method comprising: providing a skin drying topical scrub intended to be applied to skin that has been in contact with the source of urushiol to remove the urushiol from the skin, the topical scrub consisting essentially of: at least one anionic surfactant and at least one nonionic surfactant selected to form a complex with the urushiol such that removal of the surfactants from the skin removes the urushiol with the surfactants and causes a drying of the skin upon which the surfactants are applied; one or more granular materials configured to roughen skin that has been in contact with urushiol and provide a sensation on the skin that causes at least some relief of the itching caused by the urushiol upon a scrubbing application of the topical scrub; and one or more counterirritants, whereby the one or more surfactants is used in removing the urushiol from the skin and the one or more counterirritants provide a sensation on the skin to which the topical scrub has been applied; scrubbing the skin with the topical scrub to cause a roughening abrasion of the skin, penetration of the surfactants into the skin to form a complex with the urushiol, and contact between the skin and the counterirritants such that the counterirritants provide a sensation to the skin, and removing the topical scrub from the skin having the rash by applying a fluid to the skin upon which the topical scrub has been applied, whereby removing the topical scrub from the skin removes urushiol from the skin and dries the skin. The use of alkalizing agents, humectants, detergents, wetting agents, antibacterial agents, stabilizers, emulsifiers, antifungal agents, colorants, moisturizers, thickening agents, preservatives, fillers, keratolytic agents, protectant agents, vasoconstrictor agents, astringent agents, local anesthetic agents, analgesic/anesthetic/antipruritic agents, antiviral agents, and wound healing agents, counterirritants along with the surfactants are claimed to remove the source of the urushiol. A limitation of this patent application is that it is focused on the removal of urushiol following exposure to minimize the subsequent dermatitis and pruritus, but it does not mechanistically reduce dermatitis and pruritus once these symptoms have appeared.

Current therapeutic approaches to dermatitis and pruritus are limited to supportive roles and are not directed against mechanistic targets. Current therapies focus on the removal of the allergen, steroid treatment against inflammation, local anesthetics, and over-the-counter (OTC) remedies against pruritus with little proven efficacy. Liu et al. (2013) showed that TRPA1-deficient mice exhibited diminished edema formation, inflammation, skin barrier damage, and scratching behavior when challenged with urushiol, suggesting inhibition of TRPA1. Inhibiting TRPA1 may be a novel strategy to interfere with the disease process of poison ivy-induced contact dermatitis.

Antihistamines are drugs that are often used to treat allergic conditions. They work by blocking the effects of histamine to provide relief and are limited to conditions associated with a histamine response. Atopic eczema (a form of atopic dermatitis) is a histamine problem, but antihistamines are reported to only have some secondary symptomatic some effects. The sedating effect is helpful because scratching makes eczema worse. There are generally two types of oral antihistamines on the market today: first-generation antihistamines and second-generation antihistamines. First-generation drugs, such as diphenhydramine (Benadryl) and chlorpheniramine (Chlor-Trimeton), affect the brain and can cause side effects like drowsiness. These antihistamines that are beneficial as a treatment for eczema, especially if the eczema symptom of itchy skin is keeping a patient from sleeping.

Major drawbacks for orally administered drugs to treat pruritus include systemic side effects that result from their administration. For example, many antihistamines result in side effects, including dry mouth, drowsiness, dizziness, nausea and vomiting, problems with urination, blurred vision and confusion. Topical application of drugs that are applied to the skin for dermatitis and pruritus are believed to have fewer side effects compared to those orally administered. Additionally, topically applied drugs result in higher concentrations in the skin than possible with orally administered drugs. Topically applied drugs also generally result in lower systemic plasma concentrations than orally administered drugs. Therefore, topically applied drugs will reach tissue concentrations that inhibit diseases or symptoms locally in the specific organs or tissues, thereby reducing the systemic exposure of the drug and the incidence of systemic side effects of the drug.

The two primary strategies targeted to control and treat pain have concentrated on preventing the propagation of action potentials in peripheral nociceptors from reaching the central nervous system, and identifying and then inhibiting the receptors whose activation will result in the generation of said action potentials. TRPA1 and Transient Receptor Potential Vanilloid 1 (TRPV1) are two key nociceptors. Similarly, TRPA1 and TRPV1 are pruriceptors that are logical targets to treat pruritus and other non-histamine dermatitis conditions.

In contrast to TRPV1 and TRPA1 which mediate the sensations of pain and itch, activation of TRPM8 by moderate cooling or menthol inhibits pain and itch. These findings support the concept that TRPM8 offers an excellent target receptor for mediating the antipruritic (as well as the analgesic) effects. Recently, Denda, M. et al, (2010) reported that TRPM8 in epidermal keratinocytes played a role in epidermal permeability barrier homeostasis and epidermal proliferation after barrier insult. They also reported than that topical application of TRPA1 agonists accelerated the recovery of epidermal permeability barrier function after disruption. They also report a significant and pronounced anti-pruritic effect in when 1-menthol was topically applied comparison with the baseline application of histamine-induced pruritus.

U.S. Pat. No. 9,439,895 discloses a method of treating a mammal, including humans, dogs, horses and cats for non-histaminic pruritus, comprising orally administering a therapeutically effective amount of RS-norketotifen or a pharmaceutically acceptable salt thereof, thereby reducing the desire to scratch in the mammal, wherein the non-histaminergic pruritus is resistant to treatment with selective histamine-receptor inhibitors, histamine H-2-receptor inhibitors and histamine H-4-receptor inhibitors, and wherein the mammal is suffering from diabetes mellitus. Ketotifen is an antimalarial prodrug of norketotifen with blood schizonticidal and liver-stage efficacy. It is taught that the inhibition of the MAS-related G protein-coupled receptors (MrgprA3)/TRPA1 axis may be a clinically relevant non-histaminergic mode of action of norketotifen.

In a similar patent to U.S. Pat. No. 9,439,895, U.S. Pat. No. 9,345,697 discloses methods of treating a mammal in need of treatment for non-histaminic pruritus by administering to the mammal in need thereof a therapeutically effective amount of RS-norketotifen or a pharmaceutically acceptable salt thereof, thereby reducing the desire to scratch in the mammal. Non-histaminergic types of pruritus are resistant to treatment with selective histamine H-1-, H-2- and H-4-receptor inhibitors. In certain aspects, the non-histaminergic pruritus is associated with a dermal disorder, a psychological disorder, a mental disorder, a nerve disorder, or a systemic disorder.

U.S. Pat. No. 9,044,510

discloses a method of treating pruritus in a subject in need of treatment thereof, the method comprising intrathecally administering a gastrin-releasing peptide receptor (GRPR) antagonist to the subject, such that pruritus is reduced compared to a subject that was not administered a GRPR antagonist. In this patent a method is also disclosed for treating pruritus in a subject in need of treatment thereof, the method comprising intrathecally administering to the subject a composition comprising a neurotoxin coupled to a gastrin-releasing peptide (GRP), such that the itch sensation of the patient is reduced compared to a patient that is not administered the composition. Bromm et al. (1995) also reported experimentally induced itch was relieved by application of 1-menthol in a (n=16) human trial. Interestingly, they reported that skin temperature was not lowered by menthol application and was similar to controls. Han. J. H. et al (2012) reported that daily application of a topical 1% icilin gel (a known TRMP8 agonist) relieved pruritus vulvae itching symptoms The itching symptoms were significantly reduced from 9 to 2 on a 1 to 10 scale after 7 days of treatment.

European patent application EP 2700431 A1 discloses a method for the administration of plant extracts in the blockade of TRPV1 channels. In particular, plant extracts of Mangifera indica and/or Fumaria officinalis and/or Rumex japonicas are claimed to be efficacious as skin irritation-reducing agent and as an antagonistic agent for TRPV1, Additionally, anti-inflammatory agents, anti-scarring agents, anti-psoriatic agents, anti-proliferative agents, anti-fungal agents, anti-viral agents or anti-septic agents could be added to the plant extracts using topical, transdermal or oral to treat acute pain, chronic pain, touch sensitivity, itching sensitivity, inflammation, fever, hot flashes, rheumatoid arthritis. The instant disclosure teaches the simultaneous use of natural plant extract TRPA1 and TRPV1 antagonists and is not limited to a TRPV1 antagonist as in EP 2700431 A1.

Several patents exists in which synthetic TRPA1 antagonists are claimed for treatment of pain and/or dermatitis and pruritus. U.S. Pat. No. 9,012,465 discloses a method of treating a TRPA1 mediated disorder in a subject, the method comprising administering an effective amount of a compound of formula VIII, or a pharmaceutically acceptable salt. The methods of U.S. Pat. No. 9,012,465 include treating pain by administering: (i) a combination of a selective TRPA1 antagonist and a selective TRPM8 antagonist; (ii) a combination of a selective TRPA1 antagonist, a selective TRPM8 antagonist, and one or more of a selective TRPV1 and/or TRPV3 antagonist; (iii) a cross-TRP inhibitor that antagonizes a function of TRPA1. and TRPM8; or (iv) a pain inhibitor that antagonizes a function of TRPA1, TRPM8, and one or more of TRPV1 and TRPV3. This patent is limited in that it is comprised of a synthetic “non-natural” compounds and it also teaches blocking a TRPM8 ion channel. The instant disclosure teaches activating the TRPM8 ion channel as a component in a composition and a method to treat dermatitis and pruritus.

U.S. Pat. No. 8,691,855 provides novel synthetic compounds useful as a potent antagonist for a TRPV1 isomer thereof, and pharmaceutically acceptable salts. This TRPV1 antagonist discloses the treatment of pain, inflammatory disease of the joints, neuropathies, HIV related neuropathy, nerve injury, neurodegeneration, stroke, urinary bladder hypersensitivity including urinary incontinence, cystitis, stomach duodenal ulcer, irritable bowel syndrome (IBS), inflammatory bowel disease (IBD), fecal urgency, gastroesophageal reflux disease (GERD), Crohn's disease, asthma, chronic obstructive pulmonary disease, cough, neurotic/allergic/inflammatory skin disease, psoriasis, pruritus, prurigo, irritation of skin, eye or mucous membrane, hyperacusis, tinnitus, vestibular hypersensitivity, episodic vertigo, cardiac diseases, hair growth-related disorders, rhinitis, and pancreatitis, by administering the synthetic TRPV1 antagonist in this patent. This patent is limited to sole use as a TRPV1 antagonist.

U.S. Pat. No. 8,362,025 provides synthetic compounds and compositions for treating or preventing conditions such as pain by modulating the activity of the TRPA1 channel. The compounds described herein can modulate the function of TRPA1 by inhibiting a TRPA1-mediated ion flux or by inhibiting the inward current, the outward current, or both currents mediated by TRPA1. In certain embodiments, a compound of the disclosure is conjointly administered with a non-steroidal anti-inflammatory compounds including, but are not limited to, piroxicam, diclofenac, etodolac, indomethacin, ketoralac, oxaprozin, tolmetin, naproxen, flubiprofen, fenoprofen, ketoprofen, ibuprofen, mefenamic acid, sulindac, apazone, phenylbutazone, aspirin, celecoxib and rofecoxib. In addition to TRPA1, other TRP channels have been implicated in pain reception and/or sensation in U.S. Pat. No. 8,362,025. For example, certain TRPM channels including TRPM8 have been implicated in the reception and/or sensation of pain.

Accordingly, in certain embodiments, the methods of the U.S. Pat. No. 8,362,025 include treating pain by administering: (i) a combination of a selective TRPA1 antagonist and a selective TRPM8 antagonist; (ii) a combination of a selective TRPA1 antagonist, a selective TRPM8 antagonist, and one or more of a selective TRPV1 and/or TRPV3 antagonists; (iii) a cross-TRP inhibitor that antagonizes a function of TRPA1 and TRPM8; or (iv) a pain inhibitor that antagonizes a function of TRPA1, TRPM8, and one or more of TRPV1 and TRPV3. This patent is limited in that it is comprises of a synthetic “non-natural” compound and it also discloses blocking a TRPM8 ion channel. The instant disclosure teaches activating the TRPM8 ion channel as a component in a composition and a method to treat dermatitis and pruritus.

U.S. Pat. No. 7,960,584 relates to novel compounds, isomer thereof or pharmaceutically acceptable salts thereof as vanilloid receptor (Vanilloid Receptor 1; VR1; TRPV1) antagonist; and a pharmaceutical composition for preventing or treating a disease such as pain, migraine, arthralgia, neuralgia, neuropathies, nerve injury, skin disorder, urinary bladder hypersensitiveness, irritable bowel syndrome, fecal urgency, a respiratory disorder, irritation of skin, eye or mucous membrane, stomach-duodenal ulcer, inflammatory diseases, ear disease, and heart disease. U.S. Pat. 7,960,584 is limited to sole use as a synthetic TRPV1 antagonist.

U.S. Pat. No. 6,949,582 B1 discloses a method of relieving analgesia and reducing inflammation using a cannabinoid delivery topical liniment composition containing from about 97.5% to about 99.5% by weight a 70% monohydric alcohol solution, and from about 0.5% to about 2.5% by weight of a synergistic cannabinoid mixture extracted from the female plant Cannabis sativa L, including in combination: 9-Tetrahydrocannabinol (delta-9-THC), 9-THC Propyl Analogue (THC-V), Cannabidiol (CBD), Cannabidiol Propyl Analogue (CBD-V), Cannabinol (CBN), Cannabichromene (CBC), Cannabichromene Propyl Analogue (CBC-V), Cannabigerol (CBG), terpenoids, and flavonoids. In U.S. Pat. No. 6,949,582 B1, it is taught that the liniment is applied topically, preferably by spraying, and the constituents of the mixture are absorbed through the skin and interact with cannabinoid receptors in the body and tissues of a human patient to produce therapeutic analgesic and anti-inflammatory effects, without undesirable psychotropic side effects. U.S. Publication No. 2015/0086494 A1 discloses a topical formulation comprising a Cannabis derived botanical drug product, wherein the concentration of tetrahydrocannabinol and/or cannabidiol in the topical formulation is greater than 2 milligrams per kilogram. In some embodiments of U.S. Publication No. 2015/0086494 A1, the topical formulation further comprises an analgesic agent, including methyl salicylate, codeine, morphine, methadone, pethidine, buprenorphine, hydromorphine, levorphanol, oxycodone, fentanyl, and a non-steroidal anti-inflammatory drug. The amount of the analgesic agent in the topical formulation is not particularly limited, so long as it is a therapeutically effective amount. A preferred amount is from 0.01 to 5 wt %, relative to the total amount of the topical formulation, more preferably from 0.1 to 1 wt %, relative to the total amount of the topical formulation.

Research reported by DePetrocellis, et. al. (2011) reported that some TRP channels may serve as ionotropic cannabinoid receptors, which, in the context of primary afferent nerve fibers, may contribute to inflammatory hypersensitivity or vasodilation. These results were supported by their earlier work in which they found previously that some cannabinoids activate TRPV1 (Ligresti et al, 2006) and TRPA1 (De Petrocellis et al, 2008) and antagonize TRPM8 (De Petrocellis et al, 2008). Because TRPA1 ion channel is a pain and inflammation sensor, the activation of TRPA1 by cannabinoids, including delta-9-THC and CBD is not a favorable property of cannabinoid compounds. Further because TRPM8 is an ion channel associated with pain relief and antinflammation, its blocking by cannabinoid compounds is not a favorable property of cannabinoid compounds, particularly delta-9-THC and CBD.

The present disclosure provides many advantages, which shall become apparent as described below.

### SUMMARY OF THE DISCLOSURE

This disclosure relates in part to a multifunctional topical pharmaceutical composition effective to treat dermatitis and associated pruritus comprising at least one natural plant extract TRPV1 antagonist, at least one natural plant extract is a TRPA1 antagonist, and a carrier.

This disclosure also relates in part to a multifunctional topical pharmaceutical composition effective to treat dermatitis and associated pruritus comprising at least one natural plant extract TRPV1 antagonist, at least one natural plant extract is a TRPA1 antagonist, a carrier, optionally at least one natural plant extract TRPM8 agonist, optionally sodium hyaluronate, optionally a NSAID, and optionally one or more cannabinoid compounds. The composition comprises: from approximately 0.1 to approximately 25% by weight of at least one TRPV1 antagonist; from approximately 0.1 to approximately 10% by weight of at least TRPA1 antagonist; optionally from approximately 0.5 to approximately 10% by weight of at least one TRPM8 agonist; optionally from approximately 0.01 to 1.0% sodium hyaluronate; optionally from approximately 0.01 to approximately 3.00% by weight of at least one NSAID; optionally from 0.01 to approximately 1.0% of one or more cannabinoid compounds; optionally from 0.1 to approximately 2.0% of one or more antihistamine compounds; optionally from 0.1 to approximately 2.5% of one or more topical steroid compounds; optionally from 0.5 μg/mL to approximately 700 μg/mL of one or more antibiotic compounds; optionally from 0.1 to 20% of thymoquinone; and optionally from 0.1 to approximately 5.0% of one or more fungistatic compounds.

This disclosure further relates in part to a multifunctional method to treat histamine induced and non-histamine dermatitis and associated pruritus in mammals from one or more of non-atopic and atopic dermatitis (AD), contact dermatitis, allergenic contact dermatitis (ACD), psoriasis, eczema, insect bites, infestations, urticaria; nociceptive, neuropathic, neurogenic, psychogenic pruritus comprising treating with a composition comprising at least one natural plant extract TRPV1 antagonist, at least one natural plant extract is a TRPA1 antagonist, and a carrier.

This disclosure yet further relates in part to a multifunctional method to treat dermatitis and associated pruritus comprising treating with a composition comprising at least one natural plant extract TRPV1 antagonist, at least one natural plant extract is a TRPA1 antagonist, a carrier, optionally at least one natural plant extract TRPM8 agonist, optionally sodium hyaluronate, optionally a NSAID, and optionally one or more cannabinoid compounds. The composition comprises: from approximately 0.1 to approximately 25% by weight of at least one TRPV1 antagonist; from approximately 0.1 to approximately 10% by weight of at least TRPA1 antagonist; optionally from approximately 0.5 to approximately 10% by weight of at least one TRPM8 agonist; optionally from approximately 0.05 to 1.0% sodium hyaluronate; optionally from approximately 0.01 to approximately 3.00% by weight of at least one NSAID; optionally from 0.01 to approximately 1.0% of one or more cannabinoid compounds; optionally from 0.1 to approximately 2.0% of one or more antihistamine compounds; optionally from 0.1 to approximately 2.5% of one or more topical steroid compounds; optionally from 0.5 μg/mL to approximately 700 μg/mL of one or more antibiotic compounds; optionally from 0.1 to 20% of thymoquinone; and optionally from 0.1 to approximately 5.0% of one or more fungistatic compounds.

This disclosure also relates in part to multifunctional topical pharmaceutical composition effective to remove urushiol from the skin and to treat dermatitis and pruritus associated with exposure to urushiol comprising at least one natural plant extract TRPV1 antagonist, at least one natural plant extract is a TRPA1 antagonist, at least one TRPM8 agonist, a solvent and a non-ionic surfactant and a carrier.

This disclosure further relates in part to multifunctional method to remove urushiol from the skin of an individual to treat dermatitis and pruritus associated with exposure to urushiol comprising treating with a composition comprising at least one natural plant extract TRPV1 antagonist, at least one natural plant extract is a TRPA1 antagonist, at least one TRPM8 agonist, a solvent and a non-ionic surfactant and a carrier, by placing an amount the composition of the skin and scrubbing the skin to contact the skin with the composition and to remove the urushiol from the skin and then to thoroughly wash the composition containing the removed urushiol from the skin and then drying the washed skin.

This disclosure yet further relates in part to multifunctional orally consumed pharmaceutical composition effective to treat dermatitis and associated pruritus comprising at least one natural oil extract TRPV1 antagonist, at least one natural plant extract is a TRPA1 antagonist and a carrier.

This disclosure also relates in part to a multifunctional method to treat dermatitis and associated pruritus comprising treating with a composition comprising at least one natural fish oil extract TRPV1 antagonist, at least one natural plant extract is a TRPA1 antagonist, Vitamin D3, cholecalciferol and thymoquinone, optionally sodium hyaluronate, and optionally one or more cannabinoid compounds. The composition comprises: from approximately 40% to approximately 98.8% by weight of at least one TRPV1 antagonist; from approximately 0.01% to approximately 15% by weight of at least one TRPA1 antagonist; optionally from 0.01% to approximately 15% by weight of thymoquinone; from approximately 0.01% to 5.0% cholecalciferol; optionally from approximately 0.05 to 2.5% sodium hyaluronate; and optionally from 0.01 to approximately 5.0% of one or more cannabinoid compounds.

This disclosure further relates in part to a preferred composition to treat dermatitis and associated pruritus comprising at least one natural fish oil extract TRPV1 antagonist, at least one natural plant extract TRPA1 antagonist, Vitamin D3, sodium hyaluronate, thymoquinone and optionally one or more cannabinoid compounds. The composition comprises: 68.75 g fish oil containing EPA and DHA; 10.00 g by weight of the essential oil Thymus satureioides containing the TRPA1 antagonist borneol; 10.00 g by weight of the essential oil Rosmarinus officinalis (cineole type) containing the TRPA1 antagonist 1,8-cineole; 10 g by weight of the essential oil Nigella sativa contain thymoquinone; 1.25 g Cholecalciferol (Vitamin D3); 1 g sodium hyaluronate; and optionally approximately 300 mg cannabidiol.

Further objects, features and advantages of the present disclosure will be understood by reference to the following drawings and detailed description.

### BRIEF DESCRIPTION OF THE DRAWINGS

FIG. 1 shows a composition used in the manufacture of a topical cream to treat dermatitis and pruritus comprising: borneol, 1,8-cineole, Omega-3 and Omega-9 fatty acids, polyoxyethylene (20) sorbitan monolaurate, sodium polyacrylate and water for the composition in accordance with Example 1.

FIG. 2 shows a composition used in the manufacture of a topical cream to treat dermatitis and pruritus comprising: Thymus satureioides essential oil, Rosmarinus Officinalis essential oil, Linum usitatissimum oil, Prunus armeniaca oil, polyoxyethylene (20) sorbitan monolaurate, sodium polyacrylate and water in accordance with Example 2.

FIG. 3 shows a composition used in the manufacture of a topical gel to treat dermatitis and pruritus comprising: borneol, 1,8-cineole, Omega-3 and Omega-9 fatty acids, sodium polyacrylate and water in accordance with Example 3.

FIG. 4 shows a composition used in the manufacture of a topical gel to treat dermatitis and pruritus comprising: Thymus satureioides essential oil, Rosmarinus Officinalis essential oil, Linum usitatissimum oil, Prunus armeniaca oil, sodium polyacrylate and water in accordance with Example 4.

FIG. 5 shows a composition used in the manufacture of a topical cream to treat dermatitis and pruritus comprising: borneol, 1,8-cineole, 1-menthol, Omega-3 and Omega-9 fatty acids, polyoxyethylene (20) sorbitan monolaurate, sodium polyacrylate and water for the composition in accordance with Example 5.

FIG. 6 shows a composition used in the manufacture of a topical cream to treat dermatitis and pruritus comprising: Thymus satureioides essential oil, Rosmarinus Officinalis essential oil, Linum usitatissimum oil, Prunus armeniaca oil, Mentha arvensis essential oil, polyoxyethylene (20) sorbitan monolaurate, sodium polyacrylate and water in accordance with Example 6.

FIG. 7 shows a composition used in the manufacture of a topical cream to treat dermatitis and pruritus comprising: Thymus satureioides essential oil, Rosmarinus Officinalis essential oil, Linum usitatissimum oil, Prunus armeniaca oil, sodium hyaluronate, Mentha arvensis essential oil, polyoxyethylene (20) sorbitan monolaurate, sodium polyacrylate and water in accordance with Example 7.

FIG. 8 shows a composition used in the manufacture of a topical cream to treat dermatitis and pruritus comprising: borneol; 1,8-cineole; 1-menthol; an oil containing one or more of CBD, CBC and CBG; Omega-3 and Omega-9 fatty acids; polyoxyethylene (20) sorbitan monolaurate; sodium polyacrylate; and water in accordance with Example 8.

FIG. 9 shows a composition used in the manufacture of a topical cream to treat dermatitis and pruritus comprising: Thymus satureioides essential oil, Rosmarinus Officinalis essential oil, Linum usitatissimum oil, Prunus armeniaca oil, Mentha arvensis essential oil, Cannabis sativa L. essential oil and Omega-3 and Omega-9 fatty acids, polyoxy ethylene (20) sorbitan monolaurate; sodium polyacrylate; and water in accordance with Example 9.

FIG. 10 shows a composition used in the manufacture of a topical cream to treat dermatitis and pruritus comprising: borneol, 1,8-cineole, 1-menthol, a NSAID, Omega-3 and Omega-9 fatty acids, polyoxyethylene (20) sorbitan monolaurate; sodium polyacrylate; and water in accordance with Example 10.

FIG. 11 shows a composition used in the manufacture of a topical cream to treat dermatitis and pruritus comprising: Thymus satureioides essential oil, Rosmarinus Officinalis essential oil, Linum usitatissimum oil, Prunus armeniaca oil, Mentha arvensis essential oil, Gaultheria procumbens essential oil, polyoxyethylene (20) sorbitan monolaurate, sodium polyacrylate, and water in accordance with Example 11.

FIG. 12 shows a composition used in the manufacture of a topical cream to remove urushiol oil from the skin following exposure to poison ivy, poison oak and poison sumac and to treat dermatitis and pruritus comprising: borneol, 1,8-cineole, 1-menthol, d-limonene, PEG-6 Cocoa Fatty Acid surfactant, Omega-3 and Omega-9 fatty acids, polyoxyethylene (20) sorbitan monolaurate, sodium polyacrylate, hydrogenated vegetable oil beads (125μ to 750μ size range), and water in accordance with Example 12.

FIG. 13 shows a composition used in the manufacture of a topical cream to treat Acute Contact Dermatitis following exposure to poison ivy, poison oak and poison sumac comprising: borneol, 1,8-cineole, 1-menthol, sodium hyaluronate, Omega-3 and Omega-9 fatty acids, poly oxy ethylene (20) sorbitan monolaurate, sodium poly aery late, and water in accordance with Example 13.

FIG. 14 shows a composition used in the manufacture of a topical spray liquid to treat to treat dermatitis and pruritus comprising: borneol, 1,8-cineole, 1-menthol, isopropyl alcohol, water, and Omega-3 and Omega-9 fatty acids in accordance with Example 14.

FIG. 15 shows a composition used in the manufacture of a topical balm to treat to treat dermatitis and pruritus comprising: borneol, 1,8-cineole, 1-menthol, waxes and plant oils and butters, and Omega-3 and Omega-9 fatty acids in accordance with Example 15.

FIG. 16 shows a composition used in the manufacture of a topical cream to treat dermatitis, pruritus and fungal infections comprising: borneol, 1,8-cineole, 1-menthol, methyl salicylate, Omega-3 and Omega-9 fatty acids, a fungistatic compound; polyoxyethylene (20) sorbitan monolaurate, sodium polyacrylate, and water in accordance with Example 16.

FIG. 17 shows a composition used in the manufacture of a topical cream to treat dermatitis, pruritus and fungal infections comprising: Thymus satureioides essential oil, Rosmarinus Officinalis essential oil, Linum usitatissimum oil, Prunus armeniaca oil, Mentha arvensis essential oil, Gaultheria procumbens essential oil, miconazole nitrate, polyoxyethylene (20) sorbitan monolaurate, sodium polyacrylate, and water in accordance with Example 17.

FIG. 18 shows a composition used in the manufacture of an orally consumed nutritional supplement to treat dermatitis and pruritus comprising: borneol, 1,8-cineole, Omega-3 and Omega-9 fatty acids, for the composition in accordance with Example 18.

FIG. 19 shows a composition used in the manufacture of an orally consumed nutritional supplement to treat dermatitis and pruritus comprising: Thymus satureioides essential oil, Rosmarinus Officinalis essential oil, and Fish Oil in accordance with Example 19.

FIG. 20 shows a composition used in the manufacture of an orally consumed nutritional supplement to treat dermatitis and pruritus comprising: Thymus satureioides essential oil, Rosmarinus Officinalis essential oil, Fish Oil and Vitamin D3 in accordance with Example 20.

### DESCRIPTION OF THE EMBODIMENTS

Surprisingly, it has been found that the topical administration of gels, creams, balms, waxes and sprays manufactured from natural plant extracts that are TRPV1 and TRPA1 antagonists are efficacious in the reduction of dermatitis and pruritus conditions in mammals. In particular, this disclosure relates to the use of compositions comprised of natural plant extract compounds that are multifunctional TRPA1 and TRPV1 ion channel antagonists, CGRP antagonists, cannabinoid CB2 antagonists, TRPM-8 agonists, MrgprCl 1 and MrgprA3-coupled protein receptor antagonists, cytokine thymic stromal lymphopoietin receptor (TSLPR) inhibitors, other cytokine inhibitors and COX-2 inhibitors. Further, these multifunctional compositions used to treat dermatitis and pruritus were found to be efficacious with the addition of steroidal compounds, cannabinoid compounds, antibacterial or antifungal compounds. Surprising rapidly elimination of symptoms of dermatitis and pruritus with treatment by these multifunctional compositions indicates a cure of the cause of the disease rather than only the alleviation of symptoms by products currently available.

This disclosure relates in part compositions for the treatment of dermatitis, pruritus, as well as fungal and bacterial infections of the skin, which are more effective than existing compositions and products. Dermatitis and pruritus conditions and diseases that are treated with the compositions in this disclosure include, but are not limited to: atopic dermatitis (AD); allergic contact dermatitis (ACD); eczema, dyshidrotic eczema; irritant contact dermatitis; neurodermatitis; perioral dermatitis; psoriasis; insect bites, infestations (e.g. scabies, pediculosis); seborrheic dermatitis; stasis dermatitis; and urticaria (a rash of round, red welts on the skin that itch intensely, sometimes with dangerous swelling, caused by an allergic reaction, typically to specific foods, also known as hives); angioedema; histamine itch, non-histamine itch and dermal fungal and bacterial infections.

This disclosure includes compositions, methods of use and manufacture of multifunctional nutritional supplements and other orally consumed drugs that are TRPV1 and TRPA1 antagonists and preferably contain the Omega-3 fatty acids; EPA (eicosapentaenoic acid) and DHA (docosahexanenoic acid). Optionally, the oral formulations may contain one or more of the following, comprising: an Omega-9 fatty acid oleic acid (OL); an Omega-3 fatty acid a-linolenic acid (ALA); and an Omega-6 fatty acid linoleic acid (LA). Many plant and animal oils contain an Omega-9 fatty acid oleic acid (OL), an Omega-3 fatty acid a-linolenic acid (ALA), an Omega-6 fatty acid linoleic acid (LA), and an Omega-3 fatty acid eicosapentaenoic acid (EPA) DHA (docosahexanenoic acid). Preferable natural sources of an Omega-9 fatty acid oleic acid (OL), an Omega-3 fatty acid a-linolenic acid (ALA), an Omega-6 fatty acid linoleic acid (LA), and an Omega-3 fatty acid eicosapentaenoic acid (EPA) include fish, crustacean and plant sources.

This disclosure also relates in part to compositions and methods of use and manufacture of topical gels, creams, balms, waxes and sprays for the treatment of dermatitis, pruritus, as well as fungal and bacterial infections of the skin, that are more effective than existing compositions and products. Dermatitis and pruritus conditions and diseases that are treated with the compositions in this disclosure include, but are not limited to: atopic dermatitis (AD); allergic contact dermatitis (ACD); eczema, dyshidrotic eczema; irritant contact dermatitis; neurodermatitis; perioral dermatitis; psoriasis; infestations (e.g. scabies, pediculosis); seborrheic dermatitis; stasis dermatitis; and urticaria (a rash of round, red welts on the skin that itch intensely, sometimes with dangerous swelling, caused by an allergic reaction, typically to specific foods, also known as hives); angioedema; histamine itch, non-histamine itch and dermal fungal and bacterial infections.

In one embodiment of the present disclosure is a topical pharmaceutical composition effective to treat dermatitis and associated pruritus comprising at least one natural plant extract TRPV1 antagonist, at least one natural plant extract TRPA1 antagonist and a carrier. While the natural plant extract TRPV1 antagonist can be selected from any Omega-3 and Omega-9 containing fatty acid natural plant extract, preferably they are comprised of one or more fatty acids of the group comprising: an Omega-9 fatty acid oleic acid (OL); an Omega-3 fatty acid a-linolenic acid (ALA); an Omega-6 fatty acid linoleic acid (LA); and an Omega-3 fatty acid eicosapentaenoic acid (EPA). Many plant and animal oils contain an Omega-9 fatty acid oleic acid (OL), an Omega-3 fatty acid α-linolenic acid (ALA), an Omega-6 fatty acid linoleic acid (LA), and an Omega-3 fatty acid eicosapentaenoic acid (EPA). Preferable natural sources of an Omega-9 fatty acid oleic acid (OL), an Omega-3 fatty acid a-linolenic acid (ALA), an Omega-6 fatty acid linoleic acid (LA), and an Omega-3 fatty acid eicosapentaenoic acid (EPA) are fixed seed plant oils selected from one or more of the group comprising but not limited to: flaxseed oil; canola oil; perilla seed oil; sunflower oil; apricot kernel oil; black seed oil; grapeseed oil; rosehip oil; safflower oil hempseed oil; soybean oil; wheat germ oil and evening primrose oil. It is well known in the art that many more plant and animal oils comprise one or more of an Omega-9 fatty acid oleic acid (OL), an Omega-3 fatty acid a-linolenic acid (ALA), an Omega-6 fatty acid linoleic acid (LA), and an Omega-3 fatty acid eicosapentaenoic acid (EPA). This disclosure can include any oil that contains one or more of these compounds. Preferable oils are those that contain the highest concentrations of one or more of the group comprising, Omega-9 fatty acid oleic acid (OL), an Omega-3 fatty acid a-linolenic acid (ALA), an Omega-6 fatty acid linoleic acid (LA), and an Omega-3 fatty acid eicosapentaenoic acid (EPA).

In yet another embodiment of the present disclosure, the preferred natural plant extract fixed seed oils are from flaxseed oil and apricot kernel oil.

In yet another embodiment of the present disclosure are oral pharmaceutical compositions effective to treat dermatitis and associated pruritus comprising at least one natural plant, crustacean or fish extract TRPV1 antagonist, at least one natural plant extract TRPA1 antagonist and optionally a carrier. The natural plant, crustacean or fish extract TRPV1 antagonist can be selected from any Omega-3 and Omega-9 containing fatty acid natural plant extract and preferably they are comprised of one or more fatty acids of the group comprising: an Omega-9 fatty acid oleic acid (OL); an Omega-3 fatty acid a-linolenic acid (ALA); an Omega-6 fatty acid linoleic acid (LA); and an Omega-3 fatty acid eicosapentaenoic acid (EPA). Preferable natural sources of an Omega-9 fatty acid oleic acid (OL), an Omega-3 fatty acid α-linolenic acid (ALA), an Omega-6 fatty acid linoleic acid (LA) are fixed seed plant oils selected from one or more of the group comprising but not limited to: flaxseed oil; canola oil; perilla seed oil; sunflower oil; apricot kernel oil; black seed oil; grapeseed oil; rosehip oil; safflower oil hempseed oil; soybean oil; wheat germ oil and evening primrose oil. Preferable natural sources of and an Omega-3 fatty acid eicosapentaenoic acid (EPA) and docosahexaenoic acid (DHA) are from fish and crustacean oils. It is well known in the art that many more plant and animal oils comprise one or more of an Omega-9 fatty acid oleic acid (OL), an Omega-3 fatty acid α-linolenic acid (ALA), an Omega-6 fatty acid linoleic acid (LA), and an Omega-3 fatty acid eicosapentaenoic acid (EPA). This disclosure can include any oil that contains one or more of these compounds. Preferable oils are those that contain the highest concentrations of one or more of the group comprising, Omega-9 fatty acid oleic acid (OL), an Omega-3 fatty acid a-linolenic acid (ALA), an Omega-6 fatty acid linoleic acid (LA), and an Omega-3 fatty acid eicosapentaenoic acid (EPA).

In one embodiment of the present disclosure, the natural plant extract TRPA1 antagonist is selected from one or more naturally occurring compounds comprising but not limited to the group: borneol; 1,8-cineole; camphor; (−)-fenchone; fenchyl alcohol; 2-methylisoborneol and norcamphor.

In another embodiment of this present disclosure, the source of the naturally occurring TRPA1 antagonists is from one or more essential oils but not limited to the group: Inula essential oil (Inula graveolens; Thyme borneol essential oil Thymus satureioides; Gurjun Oil (Dipterocarpus Turbinatus); Camphorwood essential oil (Cinnamomum camphora Chvar. Borneol); Camphor laurel essential oil (Cinnamomum camphora); Indonesian cinnamon essential oil (Cinnamomun burmanni); Sage essential oil (Salvia Officinalis); Eucalyptus essential oil (Eucalyptus polybractea, Eucalyptus camaldulensis, Eucalyptus smithii, Eucalyptus globulus, Eucalyptus radiate, Eucalyptus spp.); Rosemary essential oil (cineole type) (Rosmarinus Officinalis); Helichrysum gymnocephalum; Bay Laurel Essential Oil (Laurus nobilis); Cannabis essential oil (Cannabis sativa, Cannabis indica, and Cannabis ruder alis); Spanish Sage essential oil (Salvia lavandulifolia).

In yet another embodiment of the present disclosure, the preferred natural plant extract TRPA1 antagonist is borneol. The preferred source of the natural plant extract TRPA1 antagonist is from Thyme borneol essential oil (Thymus satureioides). An additional source of borneol can be from a synthetic source. In addition to being a TRPA1 antagonist, borneol provides fungistatic and antibacterial properties of compositions in this instant disclosure. In this current disclosure borneol may also be included in compositions to inhibit or block IL-1 β, IL-4, IL-5, IL-6, and TNF-α.

In yet another embodiment of the present disclosure, an additional preferred natural plant extract TRPA1 antagonist is 1,8-cineole. The preferred source of the natural plant extract TRPA1 antagonist is from Rosemary essential oil (cineole type) (Rosmarinus Officinalis). In addition to being a TRPA1. antagonist, 1,8-cineole provides fungistatic properties of compositions in this instant disclosure. In this current disclosure 1,8-cineole may also be in included in compositions to block (IL)-4, IL-6, IL-8, IL-13 and IL-17A.

In another embodiment of the present disclosure includes pharmaceutical composition and a method to treat dermatitis and pruritus comprises at least one natural plant, fish or crustacean extract TRPV1 antagonist, at least one natural plant extract TRPA1 antagonist and a carrier and at least one natural plant extract TRPM8 agonist. Surprisingly, the addition of a TRPM8 agonist to this a composition of at least one natural plant extract TRPV1 antagonist and at least one natural plant extract TRPA1 antagonist to treat dermatitis and pruritus significantly increases the effectiveness of the treatment and leads to a more rapid cure of dermatitis and pruritus.

In yet another embodiment of the present disclosure the TRPM8 agonist is 1-menthol. The source of 1-menthol can be either synthetic or natural. The preferred source of 1-menthol is natural and is selected from the group of one or more essential oils of Mentha spp.: including, but not limited to, Mentha piperita; and Mentha arvensis. Because 1-menthol is also a known TRPA1 agonist in humans, (which would not be advantageous in these compositions as TRPA1 is known to be a cause of pruritus) the addition of natural plant extract TRPA1 antagonists helps to block the TRPA1 activation caused by menthol.

In another embodiment of the present disclosure further comprises sodium hyaluronate, a salt of hyaluronic acid. Hyaluronic acid has recently been discovered to be a TRPV1 antagonist associated with nociception. Sodium hyaluronate is a biotechnology manufactured natural compound that is a TRPV1 antagonist and an inhibitor of IL-Iβ, IL-8, IL-6, prostaglandin E2 (PGE2), and tumor necrosis factor (TNF-α). Sodium hyaluronate alone has been successfully used alone to treat radiation recall dermatitis and facial seborrheic dermatitis,

In yet another embodiment this present disclosure is a nonsteroidal anti-inflammatory agent (NSAID) in pharmaceutical compositions and a methods to treat dermatitis and pruritus comprising at least one natural plant extract TRPV1 antagonist, at least one natural plant extract TRPA1 antagonist, a carrier and optionally a TRPMS agonist, and optionally sodium hyaluronate. The NSAID agent can be a naturally occurring compound or one that is synthetically manufactured. Synthetically manufactured NSAIDs that are included in compositions and methods to treat dermatitis and pruritus in this disclosure can be selected from the group, but not limited to arthrotec, celecoxib, diclofenac, rofecoxib, vadecoxib, naproxen, ketoprofen, felbinac, ibuprofen, piroxicam, benzydamine, indomethacin and diclofenac.

In yet another embodiment of this present disclosure the preferred NSAID is diclofenac.

In yet another embodiment of this present disclosure a naturally occurring NSAID prodrug is methyl salicylate. While methyl salicylate is a naturally occurring compound found in the essential oil from Gaultheria procumbens (wintergreen), it can also be synthetically manufactured and used in compositions of this disclosure. Because methyl salicylate is also a known TRPA1 agonist (which would not be advantageous in these compositions as TRPA1 is known to be a cause of pruritus) the addition of natural plant extract TRPA1 antagonists helps to block the TRPA1 activation caused by methyl salicylate. Methyl salicylate also increases skin penetration of chemicals topically applied in formulations of this instant disclosure, as well as providing fungistatic properties.

In another embodiment this present disclosure cannabinoid compounds are included in pharmaceutical compositions and a methods to treat dermatitis and pruritus comprising at least one natural plant, fish or crustacean extract TRPV1 antagonist, at least one natural plant extract TRPA1 antagonist, a carrier, optionally a TRPM8 agonist, optionally sodium hyaluronate and optionally a NSAID.

In yet another embodiment of this present disclosure cannabinoid compounds are selected from one or more of the group comprising: cannabidiol (CBD), cannabigerol (CBG), cannabichromene (CBC), cannabidivarin (CBDV), and delta9-tetrahydrocannabinol (THC).

In yet another embodiment of this present disclosure preferred cannabinoid compound is cannabidiol.

In another embodiment this present disclosure is an antihistamine compound in pharmaceutical compositions and a method to treat dermatitis and pruritus comprising at least one natural plant, fish or crustacean extract TRPV1 antagonist, at least one natural plant extract TRPA1 antagonist, a carrier optionally a TRPM8 agonist, optionally sodium hyaluronate, optionally a NSAID and optionally a cannabinoid compound. Preferred anti histamines used in compositions associated with patent are diphenhydramine and chlorpheniramine, doxepin, with diphenhydramine being the most preferred.

In another embodiment this present disclosure is a steroid compound in pharmaceutical compositions and a methods to treat dermatitis and pruritus comprising at least one natural plant, fish or crustacean extract TRPV1 antagonist, at least one natural plant extract TRPA1 antagonist, a carrier optionally a TRPM8 agonist and optionally sodium hyaluronate. Steroid compounds that are included in compositions and methods to treat dermatitis and pruritus can be selected from the group comprising, but not limited to; triamcinolone; fluocinolone; prednicarbate; desonide; betamethasone; hydrocortisone; diflorasone; desoximetasone; mometasone; clobetasol;clocortolone; and halcinonide.

In yet another embodiment of this present disclosure the preferred steroid compound is hydrocortisone.

In another embodiment this present disclosure is an antibiotic compound in pharmaceutical compositions and a methods to treat dermatitis, pruritus and bacterial infections of the skin, comprising at least one natural plant, fish or crustacean extract TRPV1 antagonist, at least one natural plant extract TRPA1 antagonist, a carrier optionally a TRPM8 agonist, optionally sodium hyaluronate, and optionally a steroid. Antibiotic compounds that are included in compositions and methods to treat dermatitis and pruritus can be selected from the group comprising, but not limited to; bacitracin, chlorhexidine gluconate, mupirocin, neomycin, polymyxin B and retapamulin. In this present disclosure the preferred antibiotic is a mixture of neomycin, bacitracin and polymyxin B.

In another embodiment this present disclosure is a fungistatic compound in pharmaceutical compositions and a methods to treat dermatitis, pruritus and dermal fungal infections comprising at least one natural plant, fish or crustacean extract TRPV1 antagonist, at least one natural plant extract TRPA1 antagonist, a carrier, optionally a TRPM8 agonist, optionally methyl salicylate, and optionally an antibiotic compound. Fungistatic compounds that are included in compositions and methods to treat dermatitis, pruritus and topical fungal infections can be selected from the group comprising, but not limited to; fluconazole, itraconazole, clotrimazole, miconazole, ketoconazole, econazole, terbinafine, naftifine, butenafine and amphotericin.

In yet another embodiment of this present disclosure the preferred fungistatic compound is miconazole nitrate.

In one embodiment of the present disclosure, compositions comprise sprayable topical liquids for the treatment of dermatitis and pruritus wherein a major portion by weight of said carrier is a hydrophilic alcohol. In a preferred aspect of this present disclosure the hydrophilic alcohol is isopropyl alcohol. The sprayable liquid may be applied from a hand-pumped spray bottle or alternatively, an aerosol from an inert gas pressurized container spray. The pressurized container may comprise a single or dual bag-on-valve system to eliminate contact of the liquid with the propellant compressed gas. A person ordinarily skilled in the art would recognize and understand that all of the formulations and examples shown in this disclosure that comprise topically applied liquids that can also be sprayed, could also be made into creams, gels, liquids with the viscosity essentially of that of water, sprays, balms, waxes and massage oils and incorporated into skin patch products for the treatment of dermatitis, pruritus and bacterial and fungal skin infections.

In one embodiment of the present disclosure the composition comprises a viscous topical gel for the treatment of dermatitis and pruritus wherein the carrier forms a viscous gel comprising at least one thickening agent and water. A thickening agent can be selected from the group, but not limited to; carbomer, cacia, alginic acid, bentonite, carboxymethyl cellulose, ethylcellulose, hydroxy ethyl cellulose, hydroxypropyl cellulose, magnesium aluminum silicate (Veegum), methylcellulose, poloxamers (Pluronics), polyvinyl alcohol, sodium alginate, tragacanth, guar gum, and xanthan gum. In a preferred embodiment of this present disclosure the preferred thickener is the carbomer sodium polyacrylate. In one aspect of this present disclosure, the carrier is a viscous gel composition wherein the thickening agent is mixed with the oil phase, comprising one or more of the hydrophobic components of the composition, then mixed with water. A person ordinarily skilled in the art would recognize and understand that all of the formulations and examples shown in this disclosure that comprise gels, could also be made into creams, liquids with the viscosity essentially of that of water, sprays, balms, waxes and massage oils and incorporated into skin patch products for the treatment of dermatitis, pruritus and bacterial and fungal skin infections.

In another embodiment of the present disclosure the composition comprises a viscous topical cream for the treatment of dermatitis and pruritus wherein the carrier forms a viscous cream comprising at least one thickening agent, a surfactant and water. In one aspect of this disclosure the surfactant comprises a non-ionic surfactant. The non-ionic surfactant can be selected from a group comprising but not limited to: poly oxy ethylene (20) sorbitan monolaurate; polyoxyethylene (20) sorbitan monooleate; poly oxy ethylene (20) sorbitan monopalmitate; polyoxyethylene (20) sorbitan monostearate; sorbitan trioctadecanoate; polyglyceryl-3 stearate; polyglyceryl-3 palmitate; polyglyceryl-2 laurate; polyglyceryl-5 laurate; polyglyceryl-5 oleate; polyglyceryl-5 dioleate; and poly glyceryl-10 diisostearate. In a preferred embodiment of this present disclosure the viscous cream is comprised of the surfactant polyoxyethylene (20) sorbitan monolaurate, the thickening agent sodium polyacrylate and water. In one aspect of this present disclosure, the carrier is a viscous cream-like gel composition wherein the thickening agent and the surfactant are mixed with the oil phase, comprising one or more of the hydrophobic components of the composition, then mixed with water. For the purpose of this disclosure the gel is transparent and the cream is not transparent to light. A person ordinarily skilled in the art would recognize and understand that all of the formulations and examples shown in this disclosure that comprise creams, could also be made into gels, liquids with the viscosity essentially of that of water, sprays, balms, waxes, massage oils and incorporated into skin patch products for the treatment of dermatitis, pruritus and bacterial and fungal skin infections.

Yet another embodiment of the present disclosure the composition comprises a hydrophobic topical wax for the treatment of dermatitis and pruritus wherein the carrier forms a wax, comprising at least one wax and optionally, at least one oil and an oil butter. In one aspect of the present disclosure wax is selected from beeswax, candelilla wax, camauba wax and jojoba wax and the fixed seed oil is one or more of the group, apricot kernel oil, black seed oil; flaxseed oil, hemp oil, kiwifruit seed oil, olive oil, pumpkin seed oil sweet almond oil and walnut oil. In another aspect of this present disclosure, the oil butter is selected from cocoa butter, hemp seed butter, mango butter, olive butter, shea butter and sweet almond butter. In a preferred aspect of this present disclosure the preferred wax composition consists of beeswax at a concentration from approximately 25 to approximately 98% by weight, flax seed oil at a concentration from approximately 2 to approximately 75% by weight and hemp seed butter from about 3 to 75% by weight. A person ordinarily skilled in the art would recognize and understand that all of the formulations and examples shown in this disclosure that comprise waxes and balms, could also be made into gels, creams, liquids with the viscosity essentially of that of water, sprays, massage oils and incorporated into skin patch products for the treatment of dermatitis, pruritus and bacterial and fungal skin infections.

Yet in another embodiment of the present disclosure is a composition comprising a hydrophobic topical mixture for the treatment of dermatitis and pruritus in which the carrier is a fixed plant oil or seed oil or a mixture of fixed plant and/or seed oils to form a therapeutic massage oil. In one embodiment of the present disclosure the fixed plant and seed oils is one or more of sweet almond oil, flax seed oil, evening primrose oil, jojoba oil, apricot kernel oil, black seed oil; grape seed oil, hemp seed oil, hemp oil. In a preferred embodiment of this present disclosure the TRPV1 antagonist fixed plant oils and seed oils composition comprises sweet almond oil (29.89%), grape seed oil (40.11%), apricot kernel oil (13.04%), hemp seed oil (10.87%), evening primrose oil (2.72%) and jojoba oil (2.72%) and the TRPA1 antagonists are rosemary essential oil (0.27%) and thyme essential oil (0.27%), the TRPM8 agonist is peppermint essential oil (0.27%) and the COX-2 inhibitor is wintergreen essential oil (0.27%). The pH of healthy skin is approximately 4.7. The pH of the compositions in this present disclosure can be from pH of approximately 4.5 up to a pH of approximately 7.3. Activation of TRPM8 does not appear to be affected over this pH range. A person ordinarily skilled in the art would recognize and understand that all of the formulations and examples shown in this disclosure that comprise therapeutic massage oils, could also be made into creams, liquids with the viscosity essentially of that of water, sprays, balms, waxes and incorporated into skin patch products for the treatment of dermatitis, pruritus and bacterial and fungal skin infections.

With the exception of balms and waxes disclosed in this present disclosure, all of the compositions can be manufactured at normal ambient temperatures and pressures typically varying from 50° F. (10° C.) to 100° F. (37.8° C.), without limiting the effectiveness of the final formations.

### LITERATURE REFERENCES

Anderson, H. H., Melholt, C, Hilborg, S. D., Anne Jerwaiarz, A. J., Randers, A., and Simoni, A. (2017). Antipruritic Effect of Cold-induced and Transient Receptor Potential Agonist-Induced Counter-irritation on Histaminergic Itch in Humans. Acta Derm Venereol. 97: XX-XX, doi: 10.2340/00015555-2447. epub ahead of print.

Bautista, D. M., et al. (2006). TRPA1 mediates the inflammatory actions of environmental irritants and proalgesic agents. Cell. 124: 1269-1282.

Biro, T., Toth, B. I., Marincsak, R., Dobrosi, N., Geczy, T., and Paus, R. (2007). TRP channels as novel players in the pathogenesis and therapy of itch. Biochim. Biophys. Acta. 1772, 1004-1021.

Brenaut E, Marcorelles P, Genestet S, Menard D, Misery L.

(2015). Pruritus: an underrecognized symptom of small-fiber neuropathies. J Am Acad Dermatol. 72: 328-332.

Bromm B, Scharein E, Darsow U, Ring J. (1995). Effects of menthol and cold on histamine-induced itch and skin reactions in man. Neurosci Lett. 187: 157-160.

Caires R, Luis E, Tabemer F J, et al. (2015). Hyaluronan modulates TRPV1 channel opening, reducing peripheral nociceptor activity and pain. Nat Commun.; 6:8095.

Carstens E, Jinks S L (1998). Skin cooling attenuates rat dorsal horn neuronal responses to intracutaneous histamine. Neuroreport. 9:4145-4149.

Denda, M., Tsutsumi, M. &. Denda, S. (2010). Topical application of TRPM8 agonists accelerates skin permeability barrier recovery and reduces epidermal proliferation induced by barrier insult: role of cold-sensitive TRP receptors in epidermal permeability barrier homeostasis. Exp. Dermatol. 19, 791-795.

Fruhstorfer H, Hermanns M, Latzke L. (1986).The effects of thermal stimulation on clinical and experimental itch. Pain. 24:259-269.

Fernandes, E. S., Vong, C. T., Quek, S., Cheong, J., Awal, S., Gentry, C, Aubdool, A. A., Liang, L, Bodkin, J. V., Bevan, S., Heads, R., and Brain, S. D. (2012). Superoxide generation and leukocyte accumulation: key elements in the mediation of leukotriene B4-induced itch by transient receptor potential ankyrin 1 and transient receptor potential vanilloid 1. FASEB J. 27, 1664-1673.

Fernandez-Pena, C, Viana, F. (2013). Targeting TRPM8 for pain relief. The Open Pain Journal. (Suppl 1): 154-164.

Gualdani R, Ceruti S, Magni G, Merli D, Di Cesare Mannelli L, Francesconi O et al. (2015) Lipoic-based TRPA1/TRPV1 antagonist to treat orofacial pain. ACS Chem Neurosci. 6(3):380-5.

Han J H, Choi H K, Kim S J. (2012). Topical TRPM8 Agonist (icilin) relieved vulva pruritus originating from lichen sclerosus et atrophicus. Acta Derm Venereol. 92:561-562.

Han L, Ma C, Liu Q, et al. (2013). A subpopulation of nociceptors specifically linked to itch. Nat. Neurosci. 16(2): 174-82.

Hojland C R, Andersen H H, Poulsen J N, Arendt-Nielsen L, Gazerani P. (2015). A human surrogate model of itch utilizing the TRPA1 agonist trans-cinnamaldehyde. Acta Derm Venereol. 798-803.

Kaimoto, T., Hatakeyama, Y., Takahashi, K., Imagawa, T., Tominaga. M., and Ohta, T. (2016), Involvement of transient receptor potential Al channel in algesic and analgesic actions of the organic compound limonene. European Journal of Pain. 20 (7) 1155-1165.

Kotan, R., Kordali, S., and Cakir, A. (2007). Screening of antibacterial activities of twenty-one oxygenated monoterpenes. Z. Naturforsch. 62, 507-513.

Liu Q, Tang Z, Surdenikova L, et al.: (2009). Sensory neuron-specific GPCR Mrgprs are itch receptors mediating chloroquine-induced pruritus. Cell. 2009; 139(7): 1353-65.

Liu Q, Weng H J, Patel K N, et al. (2011). The distinct roles of two GPCRs, MrgprCl 1 and PAR2, in itch and hyperalgesia. Sci Signal. 4(181): ra45.

Liu Q, Sikand P, Ma C, et al. (2012), Mechanisms of itch evoked by β-alanine, JNeurosci. 32(42).

Liu, T., and Ji, R. R. (2012). Oxidative stress induces itch via activation of transient receptor potential subtype ankyrin 1 in mice. Neurosci.. Bull. 28, 145-154.

Liu, B., Escalera, J., Balakrishna, S., Fan, L., Caceres, A. I., Robinson, E. et al, (2013). TRPA1 controls inflammation and pruritogen responses in allergic contact dermatitis. FASEB J. 27:3549-356.

Leung, D. Y. (2000). Atopic dermatitis: new insights and opportunities for therapeutic intervention, J. Allergy Clin. Immunol. 105, 860-876.

Matta, J. A., Miyares, R. L. & Ahem, G. P. (2007). TRPV1 is a novel target for omega-3 polyunsaturated fatty acids. J. Physiol. 578, 397-411.

Mishra S K, Hoon M A: (2013). The cells and circuitry for itch responses mice. Science. 2013; 340(6135).

Morales-Lazaro et al. (2016). Inhibition of TRPV1 channels by a naturally occurring omega-9 fatty acid reduces pain and itch. Nature Communications, (7) 13092.

Perez-Faginas, P. M., Teresa Aranda, de la Torre-Martinez, R., Quirce, S., Fernandez-Carvajal, A., Ferrer-Montiel, A., Gonzalez-Muniz, R. (2016). New TRPV1, TRPM8 and TRPA1 antagonists from a single linear β,γ-diamino ester scaffold. RSCAdv. 6, 6868-6877.

Picazo-Juarez, G. et al. (2011). Identification of a binding motif in the S5 helix that confers cholesterol sensitivity to the TRPV1 ion channel. J. Biol. Chem. 286, 24966-24976.

Puntambekar, P., Van Buren, J., Raisinghani, M., Premkumar, L. S. and Ramkumar, V. (2004). Direct interaction of adenosine with the TRPV1 channel protein. J. Neurosci. 24, 3663-3671.

Qu L, Fan N, Ma C, et al. (2014). Enhanced excitability of MRGPRA3- and MRGPRD-positive nociceptors in a model of inflammatory itch and pain. Brain. 137(Pt 4): 1039-50.

Schmidt, R, M. Schmelz, C. Forster, M. Ringkamp, H. E. Torebjork, H. O. Handwerker, (1995). Novel classes of responsive and unresponsive C nociceptors in human skin, J. Neurosci. 15, 333-341.

Schmidt, R., M. Schmelz, M. Ringkamp, H. O. Handwerker, H E. Torebjork, (1997). Innervation territories of mechanically activated C nociceptor units in human skin, J. Neurophysiol. 78, 2641-2648.

Schwartz, E. S., J. H. La, N. N. Scheff, B. M. Davis, K M. Albers, and G. F. Gebhart (2013). TRPV1 and TRPA1 antagonists prevent the transition of acute to chronic inflammation and pain in chronic pancreatitis. J Neurosci. 33(5): 2060-2070.

Shiba T, Tamai T, Sahara Y, Kurohane K, Watanabe T, Imai Y (2012). Transient receptor potential ankyrin 1 activation enhances hapten sensitization in a T-helper type 2-driven fluorescein isothiocyanate-induced contact hypersensitivity mouse model. Toxicol Appl Pharmacol. 264: 370-376.

Takaishi, M.; Uchida, K.; Suzuki, Y.; Matsui, H.; Shimada, T.; Fujita, F.; Tominaga, M. (2016). Reciprocal effects of capsaicin and menthol on thermosensation through regulated activities of TRPV1 and trpm8. J. Physiol. Sci. 66, 143-155.

Than, J Y.-XL, Li, L., Hasan, R. & Zhang, X. (2013). Excitation and modulation of TRPA1, TRPV1, and TRPM8 channel-expressing sensory neurons by the pruritogen chloroquine. The Journal of Biological Chemistry, vol 288, no. 18, pp. 12818-12827.

Vriens, Joris, Bernd Nilius, and Rudi Vennekens. (2008). Herbal Compounds and Toxins Modulating TRP Channels. Current Neuropharmacology 6.1, 79-96. PMC.

Schlesinger T, Rowland Powell C. (2014). Efficacy and safety of a low molecular weight hyaluronic Acid topical gel in the treatment of facial seborrheic dermatitis final report. J Clin Aesthet Dermatol. 7(5): 15-8.

Schmelz, M. (2001). A neural pathway for itch. Nat. Neurosci. 4, 9-10.

Schmelz, M., R. Schmidt, H. O. Handwerker, H E. Torebjork. (2000) Encoding of burning pain from capsaicin-treated human skin in two categories of unmyelinated nerve fibres, Brain. 123, 560-571.

Schmelz, M., R. Schmidt, A. Bickel, H. O. Handwerker, H E. Torebjork. (1997). Specific C-receptors for itch in human skin, J. Neurosci. 17, 8003-8008.

Schmelz, M., R. Schmidt, C. Weidner, M. Hilliges, H E. Torebjork, H. O. Handwerker, (2003). Chemical response partem of different classes of C-nociceptors to pruritogens and algogens, J. Neurophysiol. 89, 2441-2448.

Togashi K, Inada H and Tominaga M. (2008). Inhibition of the transient receptor potential cation channel TRPM2 by 2-aminoethoxydiphenyl borate (2-APB). Br J Pharmacol 153: 1324-1330.

Wilson, S. R., Gerhold, K. A., Bifolck-Fisher, A., Liu, Q., Patel, K. N., Dong, X., and Bautista, D. M. (2011). TRPA1 is required for histamine-independent, Mas-related G protein-coupled receptor-mediated itch. Nat. Neurosci. 14, 595-602.

Wilson, S R, Nelson A M, Batia L, et al. (2013). The ion channel TRPA1 is required for chronic itch. J Neurosci. 33(22): 9283-94.

Yamamura, H., Ugawa, S., Ueda, T., Morita, A. & Shimada, S. (2008). TRPM8 activation suppresses cellular viability in human melanoma. Am. J. Physiol. Cell Physiol. 295, C296-301.

### PATENT REFERENCES

U.S. Patent Application No. 20150272942/Aberg; A. K. et al. Methods of treatment of Non-Histaminic pruritus. Oct. 1, 2015.

European Patent Application No. EP 2700431 A1. Haustedt, L. et al. Plant extracts for modulating TRPV1 function. Feb. 26, 2014.

U.S. Pat. No. 9,394,308. Li, Q., et al. Inhibiting the transient eceptor potential Al ion channel. Jul. 19, 2016.

U.S. Pat. No. 9,439,895. Aberg, A. K. et al. Methods of treating pruritic conditions mediated through non-histaminergic mechanisms in diabetic patients. Sep. 13, 2016.

U.S. Pat. No. 9,345,697. Aberg, A. K. et al. Methods of treatment of non-histaminic pruritus. May 24, 2016.

U.S. Pat. No. 9,193,729. Metcalf, I I I; C. A. et al. Inhibiting transient receptor potential ion channel TRPA1. Nov. 24, 2015.

U.S. Pat. No. 9,044,510. Chen, Z. F. et al. Compositions and methods for treating pruritus. Jun. 2, 2015.

U.S. Pat. No. 9,012,465. Wu, W. Compounds useful for treating disorders related to TRPA1. Apr. 21, 2015.

U.S. Pat. No. 9,006,207. Ng, H. Compositions useful for treating disorders related to TRPA1, Apr. 14, 2015.

U.S. Pat. No. 8,691,855. Woo, B. Y. et al. Compounds, isomer thereof, or pharmaceutically acceptable salts thereof as vanilloid receptor antagonist and pharmaceutical compositions containing the same. Apr. 8, 2014.

U.S. Pat. No. 8,530,487. Wu, X. Compounds useful for treating disorders related to TRPA1. Sep. 10, 2013.

U.S. Pat. No. 8,389,529. Moran. M. M. Methods and compositions for treating asthma. Mar. 5, 2013.

U.S. Pat. No. 8,362,025. Ng, H. Compositions useful for treating disorders related to TRPA1. Jan. 29, 2013.

U.S. Pat. No. 8,178,542. Moran. M M. Methods and compositions for treating pain. May 15, 2012.

U.S. Pat. No. 8,829,196. Bilodeau; M. T. TRPA1 antagonists. Sep. 9, 2014.

U.S. Pat. No. 8,163,761. Ng, H. Methods and compositions for treating disorders. Apr. 24, 2012.

U.S. Pat. No. 7,960,584. Suh, Y. G. Compounds, isomer thereof, or pharmaceutically acceptable salts thereof as vanilloid receptor antagonist; and pharmaceutical compositions containing the same. Jun. 14, 2011.

U.S. Pat. No. 7,858,570. Hare, W. Compositions and methods for removing urushiol and treating the resulting skin condition. Dec. 28, 2010.

U.S. Pat. No. 7,671,061. Methods and compositions for treating pain. Mar. 2, 2010.

U.S. Pat. No. 7,008,963. Yarbrough; W. M., et al. Urushiol induced contact dermatitis solution. Mar. 7, 2006.

U.S. Patent Application No. 20060177400 A1. Yarbrough, W. Topical formulation for the treatment of urushiol induced contact dermatitis and method for treating the same. Aug. 10, 2006.

U.S. Pat. No. 6,423,746. Yarbrough W. et al. Urushiol induced contact dermatitis and method of use. Jul. 23, 2002.

U.S. Pat. No. 6,022,565. Albert, B. M. et al. Rhus dermatitis treatment composition and method. Feb. 8, 2000.

U.S. Pat. No. 5,888,515. Albert, B. M. et al. Rhus dermatitis treatment composition and method. Mar. 30, 1999.

U.S. Pat. No. 5,859,066. Rosen, R. T. Method for the treatment of itching. Jan. 12, 1999.

U.S. Pat. No. 5,011,689. Misenko, T. P. Composition of matter and method for treating poison ivy. Apr. 30, 1991.

U.S. Pat. No. 4,499,086. Garren, F. G. Medication for treatment of poison oak irritation and inflammation. Feb. 12, 1985.

U.S. Pat. No. 4,259,318. Duhe, N. V. Poison ivy relief composition. Mar. 31, 1981.

U.S. Pat. No. 4,594,239. Pluim, Jr., A. W. Method for neutralizing offensive chemicals. Jun. 10, 1986.

U.S. Pat. No. 4,002,737. Borris, D. P. Prevention and/or treatment of poison ivy dermatitis. Jan. 11, 1977.

U.S. Pat. No. 4,032,662. Mannear, I. K. Method for the treatment of contact allergic dermatitis. Jun. 28, 1977.

U.S. Pat. No. 4,199,575. Gunther, R. E. Method of treating dermatitis venenata. Apr. 22, 1980.

The following examples illustrate the present disclosure but are not intended to limit scope of the compositions, the methods of manufacture and use of the topical analgesics as described above. Temperature are given in degrees centigrade (° C.) and unless otherwise noted all temperatures are at 25° C.

### Example 1

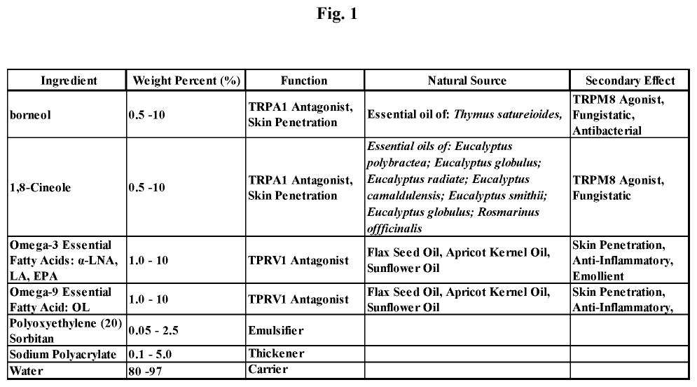

A composition and method of manufacture of a topical cream to treat dermatitis and pruritus comprising: borneol, 1,8-cineole, and Omega-3 and Omega-9 fatty acids for the composition presented in FIG. 1 consists of mixing an amount sodium polyacrylate and poly oxy ethylene (20) sorbitan monolaurate with the oil phase components of the composition to dissolve the sodium polyacrylate then adding an amount of water, such that a stable single phase homogeneous cream results. Mixing is limited to that required to dissolve the sodium polyacrylate with the oil phase components and then to mix the water for a period of time to create the stable single phase homogeneous cream and to minimize volatilization of borneol and 1,8-cineole. Methods of use of the composition of the topical cream given in this Example 1 include but are not meant to be limited to placing the cream composition in a roll-on bottle then placing the roll-on ball into the roll-on bottle container, placing the cream composition in a squeeze tube container, placing the cream composition in a hand pump bottle container and applying a therapeutic amount of the cream on the skin or wetting a patch with the cream and placing on the skin. The topical cream composition in this Example 1 can optionally be made with borneol alone or 1,8-cineole alone same total concentration range as 1,8-cineole and borneol presented in FIG. 1. In addition to treating dermatitis and pruritus, the composition also has fungistatic and antibacterial properties.

### Example 2

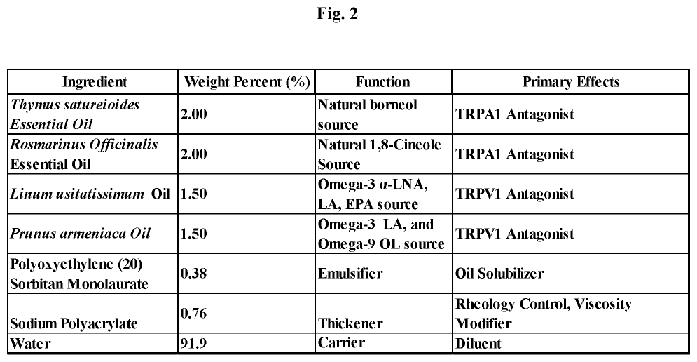

A preferred composition and method of manufacture of the topical cream to treat dermatitis and pruritus comprising: borneol, 1,8-cineole and Omega-3 and Omega-9 fatty acids is presented in this Example 2. Manufacturing consists of mixing the oil phase components comprising 20.0 g Thymus satureioides 20.0 g Rosmarinus Officinalis Essential Oil, 15.0 g Linum usitatissimum Oil and 15.0 g Prunus armeniaca with 7.6 g sodium polyacrylate, 3.8 g poly oxy ethylene (20) sorbitan monolaurate and then mixing sufficiently to dissolve the sodium polyacrylate, followed by then adding 919 g water then rapidly mixing for 2 to 5 minutes for the stable single phase homogeneous cream to form. Mixing is limited to that required to dissolve the sodium polyacrylate with the oil phase components and then to mix the water for a period of time to create the stable single phase homogeneous cream and to minimize volatilization of essential oils containing borneol and 1,8-cineole. Methods of use of the composition of the topical cream given in this Example 2 include but are not meant to be limited to placing the cream composition in a roll-on bottle then placing the roll-on ball into the roll-on bottle container, placing the cream composition in a squeeze tube container, placing the cream composition in a hand pump bottle container and applying a therapeutic amount of the cream on the skin or wetting a patch with the cream and placing on the skin. The composition of a topical cream with borneol, 1,8-cineole, menthol and Omega-3 and Omega-9 fatty acids is shown in FIG. 2. In addition to treating dermatitis and pruritus, the composition also has fungistatic and antibacterial properties.

### Example 3

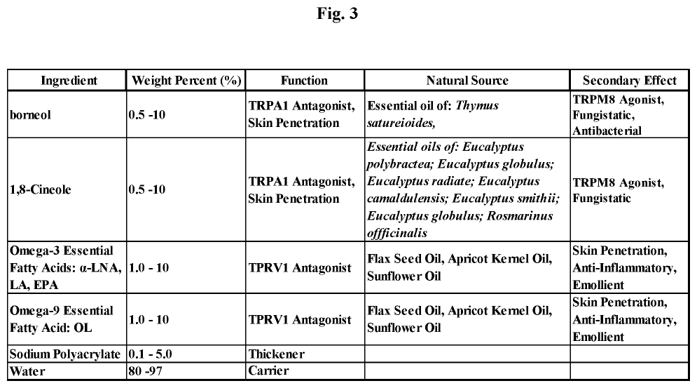

A method of manufacture of a topical gel to treat dermatitis and pruritus comprising: borneol, 1,8-cineole and Omega-3 and Omega-9 fatty acids for the composition presented in FIG. 3 consists of mixing an amount sodium polyacrylate with the oil phase components of the composition to dissolve the sodium polyacrylate then adding an amount of water, such that a stable single phase homogeneous gel results. Mixing is limited to that required to dissolve the sodium polyacryl ate with the oil phase components and then to mix the water for a period of time to create the stable single phase homogeneous gel and to minimize volatilization of borneol and 1,8-cineole. Methods of use of the composition of the topical gel given in this Example 3 include but are not meant to be limited to placing the gel composition in a roll-on bottle then placing the roll-on ball into the roll-on bottle container, placing the cream composition in a squeeze tube container, placing the cream composition in a hand pump bottle container and applying a therapeutic amount of the gel on the skin or wetting a patch with the gel and placing on the skin. The topical gel composition in this Example 3 can optionally be made with borneol alone or 1,8-cineole alone same total concentration range as 1,8-cineole and borneol presented in FIG. 3. In addition to treating dermatitis and pruritus, the composition also has fungistatic and antibacterial properties.

### Example 4

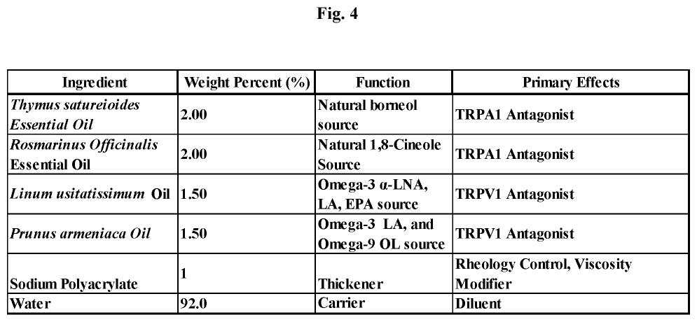

A preferred composition and method of manufacture of the topical gel to treat dermatitis and pruritus comprising: borneol, 1,8-cineole and Omega-3 and Omega-9 fatty acids is presented in this Example 4. Manufacturing consists of mixing the oil phase components comprising 20.0 g Thymus satureioides 20.0 g Rosmarinus Officinalis Essential Oil, 15.0 g Linum usitatissimum Oil and 15.0 g Prunus armeniaca with 10.0 g sodium polyacrylate and then mixing sufficiently to dissolve the sodium polyacrylate, followed by then adding 920 g water then rapidly mixing for 2 to 5 minutes for the stable single phase homogeneous gel to form. Mixing is limited to that required to dissolve the sodium polyacrylate with the oil phase components and then to mix the water for a period of time to create the stable single phase homogeneous gel and to minimize volatilization of essential oils containing borneol and 1,8-cineole. Methods of use of the composition of the topical gel given in this Example 4 include but are not meant to be limited to placing the gel composition in a roll-on bottle then placing the roll-on ball into the roll-on bottle container, placing the gel composition in a squeeze tube container, placing the gel composition in a hand pump bottle container and applying a therapeutic amount of the gel on the skin or wetting a patch with the gel and placing on the skin. The composition of a topical gel with borneol, 1,8-cineole, menthol and Omega-3 and Omega-9 fatty acids is shown in FIG. 4. In addition to treating dermatitis and pruritus, the composition also has fungistatic and antibacterial properties.

### Example 5

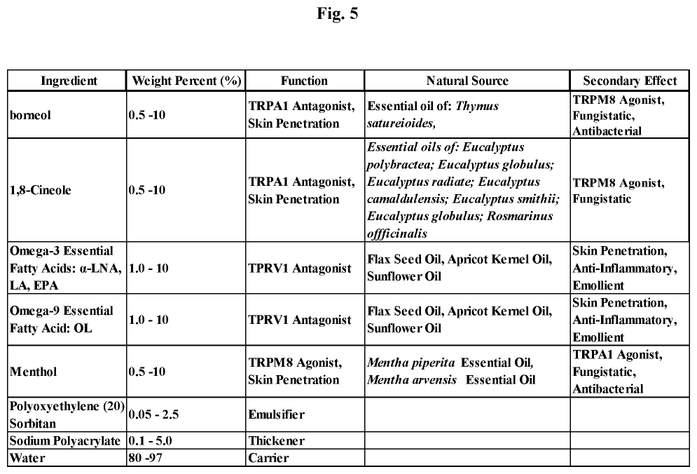

A method of manufacture of a topical cream to treat dermatitis and pruritus comprising: borneol, 1,8-cineole, 1-menthol and Omega-3 and Omega-9 fatty acids for the composition shown in FIG. 5 consists of mixing an amount sodium polyacrylate and poly oxy ethylene (20) sorbitan monolaurate with the oil phase components of the composition to dissolve the sodium polyacrylate then adding an amount of water, such that a stable single phase homogeneous cream results. Mixing is limited to that required to dissolve the sodium polyacrylate with the oil phase components and then to mix the water for a period of time to create the stable single phase homogeneous cream and to minimize volatilization of borneol, 1,8-cineole and 1-menthol. Methods of use of the composition of the topical cream given in this Example 5 include but are not meant to be limited to placing the cream composition in a roll-on bottle then placing the roll-on ball into the roll-on bottle container, placing the cream composition in a squeeze tube container, placing the cream composition in a hand pump bottle container and applying a therapeutic amount of the cream on the skin or wetting a patch with the cream and placing on the skin. The topical cream composition in this Example 5 can optionally be made with borneol alone or 1,8-cineole alone same total concentration range as 1,8-cineole, borneol and 1-menthol is shown in FIG. 5. In addition to treating dermatitis and pruritus, the composition also has fungistatic and antibacterial properties.

### Example 6

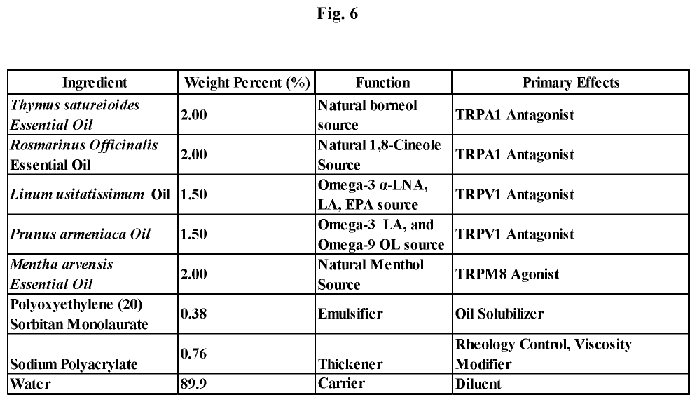

A preferred composition and method of manufacture of the topical cream to treat dermatitis and pruritus comprising: borneol, 1,8-cineole, 1-menthol and Omega-3 and Omega-9 fatty acids is presented in this Example 6. Manufacturing consists of mixing the oil phase components comprising 20.0 g Thymus satureioides 20.0 g Rosmarinus Officinalis Essential Oil, 20.0 g Mentha arvensis essential oil 15.0 g Linum usitatissimum Oil and 15.0 g Prunus armeniaca with 7.6 g sodium polyacrylate, 3.8 g polyoxy ethylene (20) sorbitan monolaurate and then mixing sufficiently to dissolve the sodium polyacrylate, followed by then adding 899 g water then rapidly mixing for 2 to 5 minutes for the stable single phase homogeneous cream to form. Mixing is limited to that required to dissolve the sodium polyacrylate with the oil phase components and then to mix the water for a period of time to create the stable single phase homogeneous cream and to minimize volatilization of essential oils containing borneol, 1,8-cineole and 1-menthol. Methods of use of the composition of the topical cream given in this Example 6 include but are not meant to be limited to placing the cream composition in a roll-on bottle then placing the roll-on ball into the roll-on bottle container, placing the cream composition in a squeeze tube container, placing the cream composition in a hand pump bottle container and applying a therapeutic amount of the cream on the skin or wetting a patch with the cream and placing on the skin. The composition of a topical cream with borneol, 1,8-cineole, 1-menthol and Omega-3 and Omega-9 fatty acids is shown in FIG. 6. In addition to treating dermatitis and pruritus, the composition also has fungistatic and antibacterial properties.

### Example 7

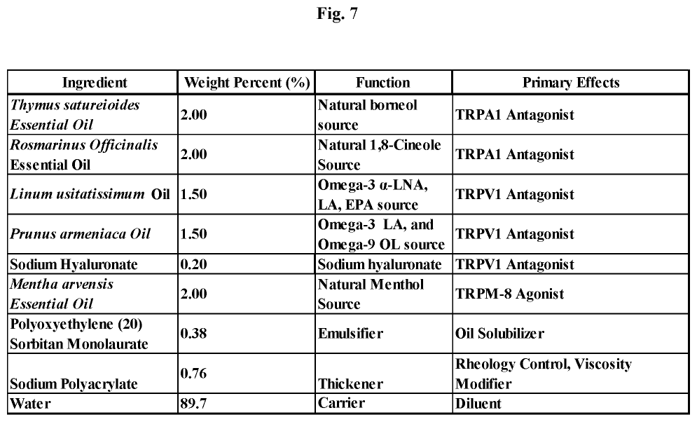

A preferred composition and method of manufacture of the topical cream to treat dermatitis and pruritus comprising: borneol, 1,8-cineole, 1-menthol, sodium hyaluronate and Omega-3 and Omega-9 fatty acids is presented in this Example 7. Manufacturing consists of mixing the oil phase components comprising 20.0 g Thymus satureioides 20.0 g Rosmarinus Officinalis Essential Oil, 20.0 g Mentha arvensis essential oil 15.0 g Linum usitatissimum Oil and 15.0 g Prunus armeniaca with 7.6 g sodium polyacrylate, 3.8 g polyoxy ethylene (20) sorbitan monolaurate and then mixing sufficiently to dissolve the sodium polyacrylate, followed by then adding 898 g cold water in which 0.75 g sodium hyaluronate has been previously dissolved, then rapidly mixing for 2 to 5 minutes for the stable single phase homogeneous cream to form. Mixing of the cold water phase containing sodium hyaluronate must be sufficient in intensity and duration to fully dissolve the sodium hyaluronate powder. Following this step mixing of the water phase containing the prior dissolved sodium hyaluronate with the oil phase components containing the prior dissolved sodium polyacrylate and the polyoxyethylene (20) sorbitan monolaurate, is required for a period of time (generally 2 to 5 minutes) to create the stable single phase homogeneous cream and to minimize volatilization of essential oils containing borneol, 1,8-cineole and 1-menthol. Methods of use of the composition of the topical cream given in this Example 7 include but are not meant to be limited to placing the cream composition in a roll-on bottle then placing the roll-on ball into the roll-on bottle container, placing the cream composition in a squeeze tube container, placing the cream composition in a hand pump bottle container and applying a therapeutic amount of the cream on the skin or wetting a patch with the cream and placing on the skin. The composition of a topical cream with borneol, 1,8-cineole, 1-menthol, sodium hyaluronate and Omega-3 and Omega-9 fatty acids is shown in FIG. 7. In addition to treating dermatitis and pruritus, the composition also has fungistatic and antibacterial properties.

### Example 8

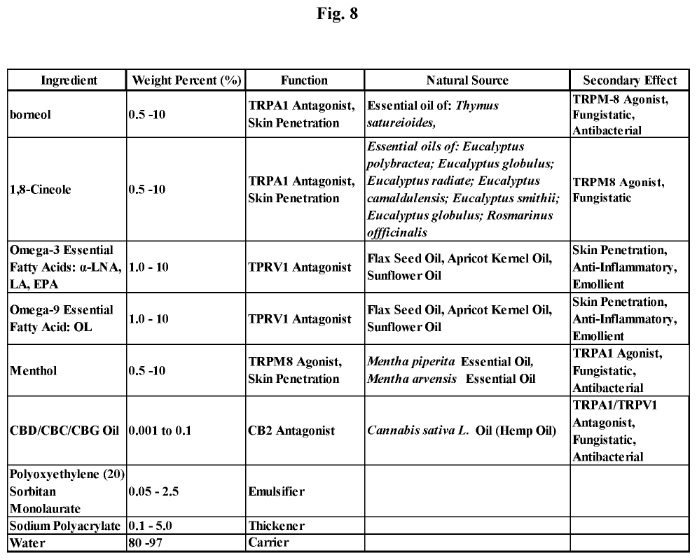

A method of manufacture of a topical cream to treat dermatitis and pruritus comprising: borneol, 1,8-cineole, 1-menthol, Omega-3 and Omega-9 fatty acids and one or more cannabinoid compounds selected from the group comprising: CBD, CBC and CBG, for the composition shown in FIG. 8. Oil containing one or more of the cannabinoid compounds; CBD, CBC and CBG is mixed with the oil phase ingredients until the CBD, CBC and CBG oil is dissolved. The following step consists of an amount sodium polyacrylate and polyoxyethylene (20) sorbitan monolaurate with the oil phase components of the composition to dissolve the sodium polyacrylate then adding an amount of water, such that a stable single phase homogeneous cream results. Mixing is limited to that required to initially dissolve the CBD, CBC and CBG oil with the other oil phase ingredients, following dissolution of the sodium polyacrylate with the oil phase components and then to mix the water for a period of time to create the stable single phase homogeneous cream and to minimize volatilization of borneol, 1,8-cineole and 1-menthol. Methods of use of the composition of the topical cream given in this Example 8 include but are not meant to be limited to placing the cream composition in a roll-on bottle then placing the roll-on ball into the roll-on bottle container, placing the cream composition in a squeeze tube container, placing the cream composition in a hand pump bottle container and applying a therapeutic amount of the cream on the skin or wetting a patch with the cream and placing on the skin. The topical cream composition in this Example 8 can optionally be made with borneol alone or 1,8-cineole alone same total concentration range as 1,8-cineole, borneol and 1-menthol is shown in FIG. 8. In addition to treating dermatitis and pruritus, the composition also has fungistatic and antibacterial properties.

### Example 9

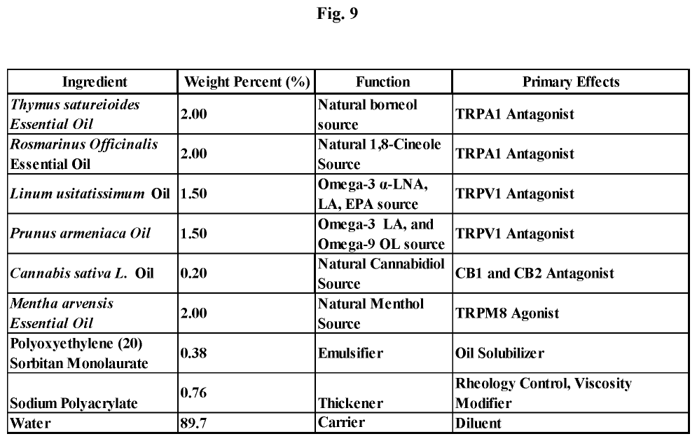

A preferred composition and method of manufacture of the topical cream to treat dermatitis and pruritus comprising: borneol, 1,8-cineole, 1-menthol, Omega-3 and Omega-9 fatty acids and one or more cannabinoid compounds selected from the group comprising CBD, CBC and CBG present in Cannabis sativa L. oil for the composition shown is shown in this Example 9. Manufacturing consists of mixing the oil phase components comprising 20.0 g Thymus satureioides essential oil, 20.0 g Rosmarinus Officinalis Essential Oil, 20.0 \hat{ }Mentha arvensis essential oil 15.0 g Linum usitatissimum Oil and 15.0 g Prunus armeniaca and 0.20 g Cannabis sativa L. oil with 7.6 g sodium polyacrylate, 3.8 g poly oxy ethylene (20) sorbitan monolaurate and then mixing sufficiently to dissolve the sodium polyacrylate, followed by then adding 897 g cold water then rapidly mixing for 2 to 5 minutes for the stable single phase homogeneous cream to form. This composition provides approximately 50 mg/kg of actual CBD, CBC and CBG compounds. Other type of CBD oils with higher or lower concentrations of CBD, CBC and CBG in the Cannabis sativa L. oil can be added to this composition to result compositions similar to that in this Example 9 that have greater or lower CBD, CBC and CBG concentrations. Mixing of the oil phase ingredients to which the Cannabis sativa L. oil has been added must take place for a long enough period to completely dissolve Cannabis sativa L. oil. Mixing of the oil phase components with the sodium polyacrylate is limited to that required to dissolve the sodium polyacrylate with the oil phase components. Following this step mixing of the water phase with the oil phase components containing the prior dissolved sodium polyacrylate and the poly oxy ethylene (20) sorbitan monolaurate, is required for a period of time (generally 2 to 5 minutes) to create the stable single phase homogeneous cream and to minimize volatilization of essential oils containing borneol, 1,8-cineole and 1-menthol. Methods of use of the composition of the topical cream given in this Example 9 include but are not meant to be limited to placing the cream composition in a roll-on bottle then placing the roll-on ball into the roll-on bottle container, placing the cream composition in a squeeze tube container, placing the cream composition in a hand pump bottle container and applying a therapeutic amount of the cream on the skin or wetting a patch with the cream and placing on the skin. The composition of a topical cream with borneol, 1,8-cineole, 1-menthol, Cannabis sativa L. oil and Omega-3 and Omega-9 fatty acids is shown in FIG. 9. In addition to treating dermatitis and pruritus, the composition also has fungistatic and antibacterial properties.

### Example 10

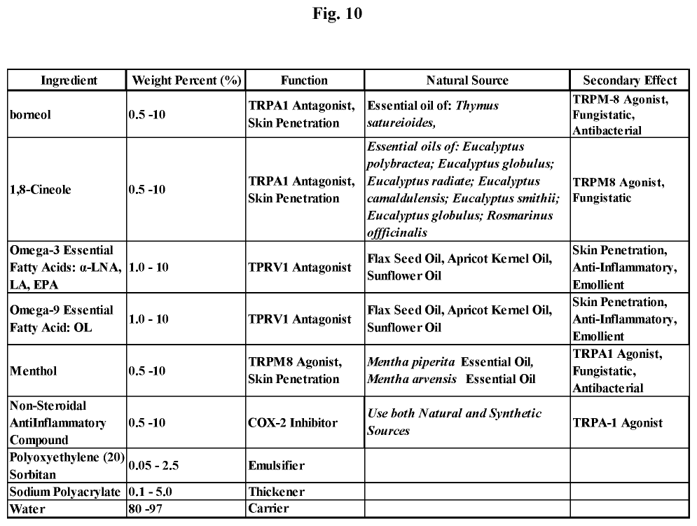

A method of manufacture of a topical cream to treat dermatitis and pruritus comprising: borneol, 1,8-cineole, 1-menthol, a NSAID and Omega-3 and Omega-9 fatty acids for the composition shown in this Example 10 consists of mixing an amount sodium polyacrylate and poly oxy ethylene (20) sorbitan monolaurate with the oil phase components of the composition to dissolve the sodium polyacrylate then adding an amount of water, such that a stable single phase homogeneous cream results. Mixing is limited to that required to dissolve the sodium polyacrylate with the oil phase components and then to mix the water for a period of time to create the stable single phase homogeneous cream and to minimize volatilization of borneol, 1,8-cineole, 1-menthol and the NSAID, if it is a volatile compound. Methods of use of the composition of the topical cream given in this Example 10 include but are not meant to be limited to placing the cream composition in a roll-on bottle then placing the roll-on ball into the roll-on bottle container, placing the cream composition in a squeeze tube container, placing the cream composition in a hand pump bottle container and applying a therapeutic amount of the cream on the skin or wetting a patch with the cream and placing on the skin. The topical cream composition in this Example 10 can optionally be made with borneol alone or 1,8-cineole alone same total concentration range as 1,8-cineole, borneol and 1-menthol is shown in FIG. 10. In addition to treating dermatitis and pruritus, the composition also has fungistatic and antibacterial properties.

### Example 11

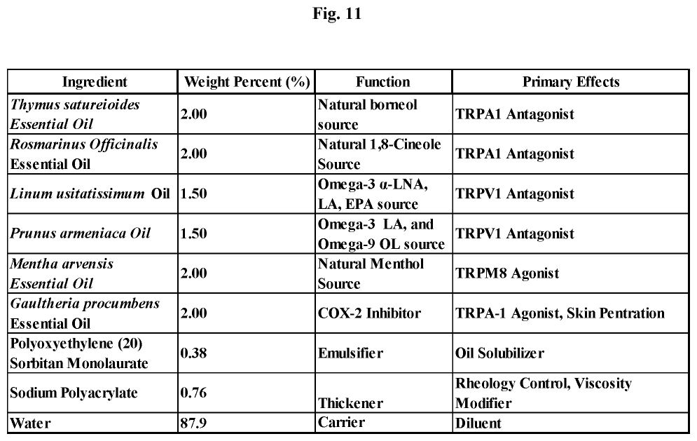

A preferred composition and method of manufacture of the topical cream to treat dermatitis and pruritus comprising: borneol, 1,8-cineole, 1-menthol, Omega-3 and Omega-9 fatty acids and the NSAID compound methyl salicylate is shown in this Example 11. Manufacturing consists of mixing the oil phase components comprising 20.0 g Thymus satureioides essential oil, 20.0 g Rosmarinus Officinalis Essential Oil, 20.0 g Mentha arvensis essential oil 15.0 g Linum usitatissimum Oil, 15.0 g Prunus armeniaca and 20.00 g Gaultheria procumbens Essential Oil with 7.6 g sodium polyacrylate, 3.8 g polyoxyethylene (20) sorbitan monolaurate and then mixing sufficiently to dissolve the sodium polyacrylate, followed by then adding 879 g cold water then rapidly mixing for 2 to 5 minutes for the stable single phase homogeneous cream to form. Mixing of the oil phase components with the sodium polyacrylate is limited to that required to dissolve the sodium polyacrylate with the oil phase components. Following this step mixing of the water phase with the oil phase components containing the prior dissolved sodium polyacrylate and the polyoxyethylene (20) sorbitan monolaurate, is required for a period of time (generally 2 to 5 minutes) to create the stable single phase homogeneous cream and to minimize volatilization of essential oils containing borneol, 1,8-cineole, 1-menthol and methyl salicylate. Methods of use of the composition of the topical cream given in this Example 11 include but are not meant to be limited to placing the cream composition in a roll-on bottle then placing the roll-on ball into the roll-on bottle container, placing the cream composition in a squeeze tube container, placing the cream composition in a hand pump bottle container and applying a therapeutic amount of the cream on the skin or wetting a patch with the cream and placing on the skin. The composition of a topical cream with borneol, 1,8-cineole, 1-menthol, Gaultheria procumbens Essential Oil and Omega-3 and Omega-9 fatty acids is shown in FIG. 11. In addition to treating dermatitis and pruritus, the composition also has fungistatic and antibacterial properties.

### Example 12

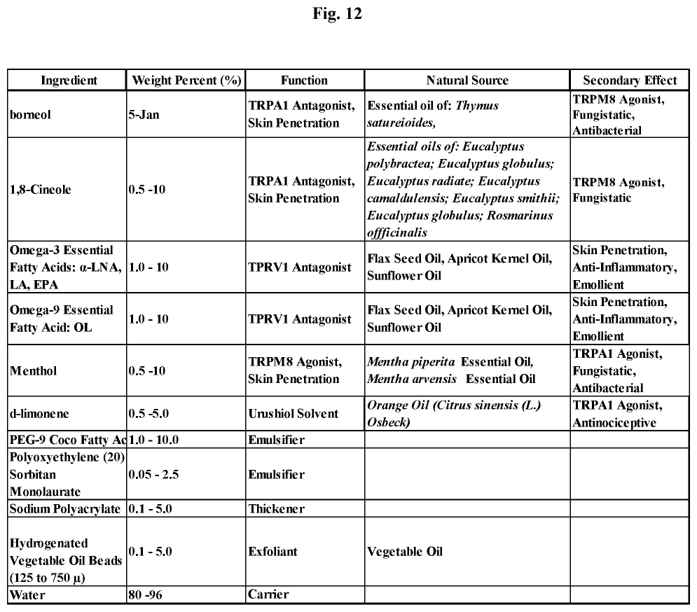

A composition and a method of manufacture of a topical cream, optionally with microbeads in the size range of 125μ to 750μ, to remove urushiol oil associated with recent exposure to poison ivy, poison oak or poison sumac comprising: borneol, 1,8-cineole, 1-menthol, d-limonene, Omega-3 and Omega-9 fatty acids and PEG-9 Cocoa Fatty Acid non-ionic surfactant, and optionally 125μ to 750μ microbeads for the composition shown in this Example 12 consists of mixing an amount sodium polyacrylate and polyoxy ethylene (20) sorbitan monolaurate with the oil phase components of the composition to dissolve the sodium polyacrylate then adding an amount of water, such that a stable single phase homogeneous cream results. The 125μ to 750μ sized microbeads can be added immediately after the water is added to the oil ingredients during the mixing process. The d-limonene and the PEG-9 Cocoa Fatty Acid should be mixed together prior to adding to the oil phase. Mixing is limited to that required to dissolve the sodium polyacrylate with the oil phase components and then to mix the water and the microbeads for a period of time to create the stable single phase heterogeneous cream and to minimize volatilization of borneol, 1,8-cineole, 1-menthol and d-limonene. Methods of use of the composition of the topical cream given in this Example 12 include but are not meant to be limited to placing the cream composition in a squeeze tube container and placing the cream composition in a hand pump bottle container and applying a quantity of the cream to preferably skin in areas where exposure to poison ivy, oak or sumac has been expected. The cream should be rubbed onto the skin for at least 1 minute and then thoroughly rinsed off with cool water. If exposure to poison ivy, oak or sumac has resulted in Acute Contact Dermatitis (ACD) when treatment with the composition of this Example 12 commenced, the same treatment regime as described above should still be followed. This procedure can be repeated up to 3 times per day for a one day period to insure removal of the urushiol oil from the skin. The topical cream composition in this Example 12 can optionally be made with borneol alone or 1,8-cineole alone same total concentration range as 1,8-cineole, borneol and 1-menthol is shown in FIG. 12. Following removal of the urushiol oil with the formulation disclosed in FIG. 12, treatment to treat reduce ACD symptoms and pruritus can be made with compositions disclosed in Examples 1 through 11 and preferably Example 13. In addition to treating dermatitis and pruritus, the composition also has fungistatic and antibacterial properties.

### Example 13

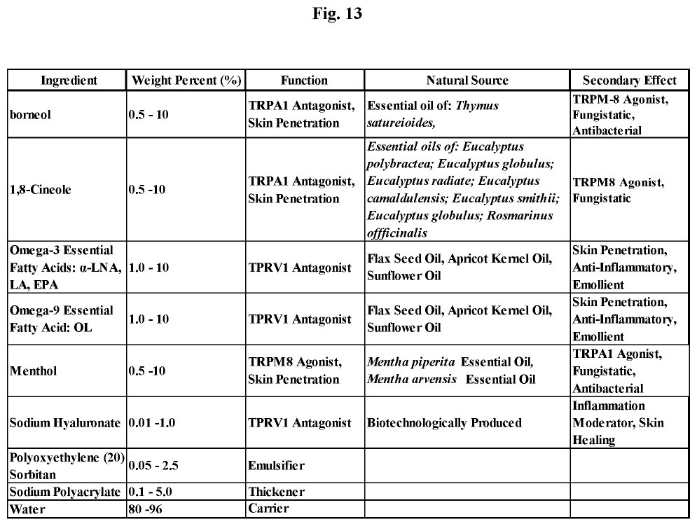

A composition and a method of manufacture of a topical cream, treat ACD and pruritus associated with prior exposure to poison ivy, poison oak or poison sumac comprising: borneol, 1,8-cineole, 1-menthol, sodium hyaluronate and Omega-3 and Omega-9 fatty acids for the composition shown in this Example 13 consists of mixing an amount sodium polyacrylate and poly oxy ethylene (20) sorbitan monolaurate with the oil phase components of the composition to dissolve the sodium polyacrylate then adding an amount of water, such that a stable single phase homogeneous cream results. Prior to the addition of water to the oil phase ingredients the sodium hyaluroate is dissolved in an amount of cold water and mixed until the sodium hyaluronate is completely dissolved in the water. Mixing is limited to that required to dissolve the sodium polyacrylate with the oil phase components and then to mix the water for a period of time to create the stable single phase homogeneous cream and to minimize volatilization of borneol, 1,8-cineole, 1-menthol. Methods of use of the composition of the topical cream given in this Example 13 to treat dermatitis and pruritus associated with prior exposure to poison ivy, oak and sumac include but are not meant to be limited to placing the cream composition in a roll-on bottle then placing the roll-on ball into the roll-on bottle container, placing the cream composition in a squeeze tube container, placing the cream composition in a hand pump bottle container and applying a therapeutic amount of the cream on the skin or wetting a patch with the cream and placing on the skin. The topical cream composition in this Example 13 can optionally be made with borneol alone or 1,8-cineole alone same total concentration range as 1,8-cineole, borneol and 1-menthol is shown in FIG. 13. In addition to treating dermatitis and pruritus, the composition also has fungistatic and antibacterial properties.

A combined method of treatment for the exposure to poison ivy, oak or sumac is a two-step process wherein the first step is to remove the urushiol oil with the composition described in Example 12, followed by the treatment of the ACD and pruritus associated with exposure to urushiol oil by the composition described in this Example 13.

### Example 14

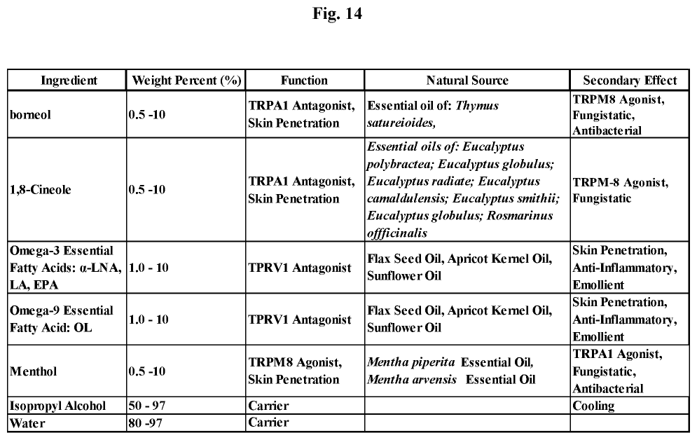

A composition and a method of manufacture of a topical spray to treat dermatitis and pruritus comprising: borneol, 1-menthol and Omega-3 and Omega-9 fatty acids for the composition shown in this Example 14 consists of mixing isopropyl alcohol with the oil phase components of the composition such that a stable single phase homogeneous liquid results, followed by the addition of an amount of water. Mixing is limited to that required to dissolve alcohol with the oil phase components and then to mix the water for a period of time to create the stable single phase homogeneous liquid and to minimize volatilization of borneol, 1,8-cineole, and 1-menthol. Methods of use of the composition of the topical liquid spray given in this Example 14 include but are not meant to be limited to sprayable liquid may be applied from a hand-pumped spray bottle or alternatively, or an aerosol from an inert gas pressurized container spray and applying a therapeutic amount of the liquid on the skin or wetting a patch with the liquid placing on the skin. The pressurized container may comprise a single or dual bag-on-valve system to eliminate contact of the liquid with the propellant compressed gas. The topical liquid spray composition in this Example 14 can optionally be made with borneol alone or 1,8-cineole alone same total concentration range as 1,8-cineole, borneol and 1-menthol is shown in FIG. 14. In addition to treating dermatitis and pruritus, the composition also has fungistatic and antibacterial properties.

### Example 15

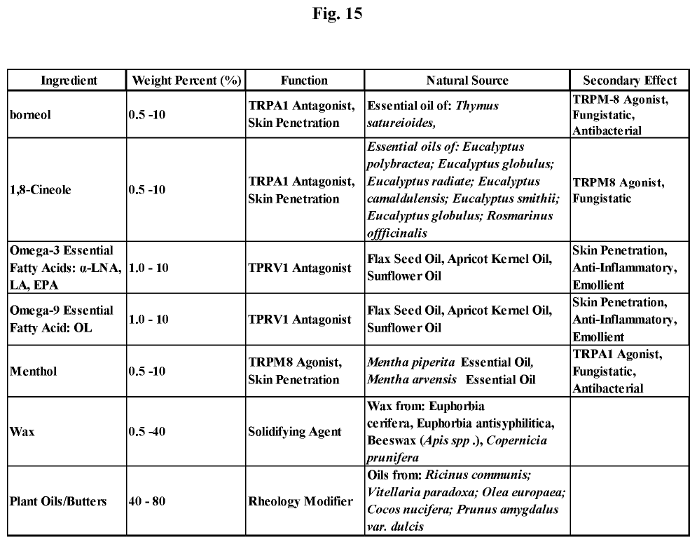

A composition and a method of manufacture of a topical balm to treat dermatitis and pruritus comprising: borneol, 1,8-cineole, 1-menthol and Omega-3 and Omega-9 fatty acids for the composition shown in this Example 15 consists of mixing an amount wax and oils associated with the fatty acid and optionally additional plant oils and plant butters and heating to or slightly above the boiling point of the waxes used. Once the wax and plant oil/butters have melted and a homogeneous melted liquid results, the essential oil compounds can be added while mixing the solution prior to its solidification and a homogeneous solution of the wax, plant oil/butters and essential oils will exist. Mixing is limited to that required to dissolve melted waxes and the plant oils/butters during heating and then when the essential oils are added to the melted wax and plant oils/butters liquid to minimize volatilization of borneol, 1,8-cineole, 1-menthol. Methods of use of the composition of the topical balm given in this Example 15 include but are not meant to be limited to application from ajar, a lip balm tube, an aluminum ointment tube or other types of sealable containers and applying a therapeutic amount of the balm on the skin, lips or wetting a patch with the balm and placing on the skin. The topical balm composition in this Example 15 can optionally be made with borneol alone or 1,8-cineole alone same total concentration range as 1,8-cineole, borneol and 1-menthol is shown in FIG. 15. In addition to treating dermatitis and pruritus, the composition also has fungi static and antibacterial properties.

### Example 16

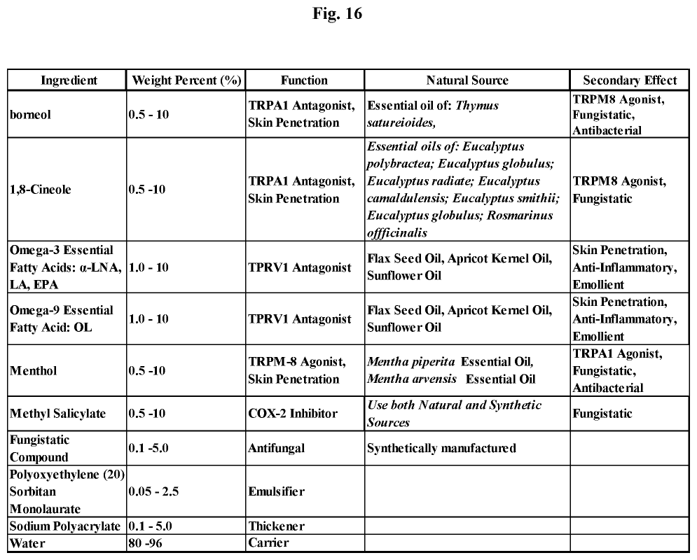

A method of manufacture of a topical cream to treat dermatitis, pruritus and fungal infections comprising: borneol, 1,8-cineole, 1-menthol, a fungi static compound and Omega-3 and Omega-9 fatty acids for the composition shown in Example 16 consists of mixing an amount sodium polyacrylate and polyoxyethylene (20) sorbitan monolaurate with the oil phase components of the composition to dissolve the sodium polyacrylate then adding an amount of a fungistatic compound to the oil phase and mixing sufficiently to dissolve the fungistatic compound in the oil, then adding an amount of water, such that a stable single phase homogeneous cream results. Mixing is limited to that required to dissolve the sodium polyacrylate and the fungistatic compound with the oil phase components and then to mix the water for a period of time to create the stable single phase homogeneous cream and to minimize volatilization of borneol, 1,8-cineole and 1-menthol if it is a volatile compound. Methods of use of the composition of the topical cream given in this Example 16 include but are not meant to be limited to placing the cream composition in a roll-on bottle then placing the roll-on ball into the roll-on bottle container, placing the cream composition in a squeeze tube container, placing the cream composition in a hand pump bottle container and applying a therapeutic amount of the cream on the skin or wetting a patch with the cream and placing on the skin. The topical cream composition in this Example 16 can optionally be made with borneol alone or 1,8-cineole alone same total concentration range as 1,8-cineole, borneol and 1-menthol is shown in FIG. 16. In addition to treating dermatitis, pruritus and fungal infections, the composition also has antibacterial properties. In addition to the compounds added to the composition shown in FIG. 16, one or more additional antibacterial compounds can be added to increase the antibacterial properties of the cream.

### Example 17

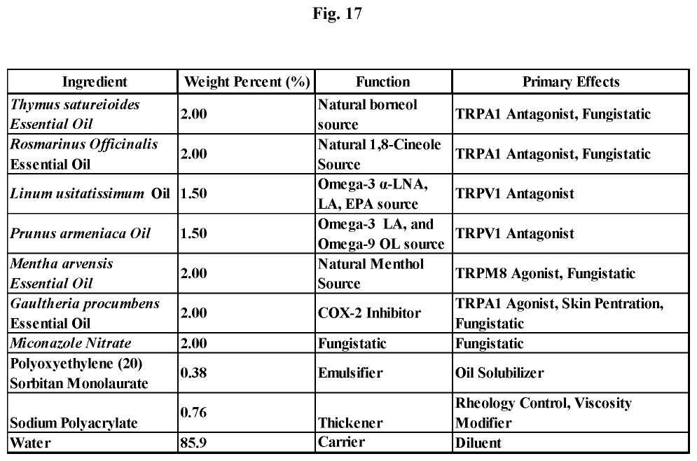

A preferred composition and method of manufacture of the topical cream to treat dermatitis, pruritus and fungal infections comprising: borneol, 1,8-cineole, 1-menthol, methyl salicylate, Omega-3 and Omega-9 fatty acids and miconazole nitrate is shown in this Example 17. Manufacturing consists of mixing the oil phase components comprising 20.0 g Thymus satureioides essential oil, 20.0 g Rosmarinus Officinalis Essential Oil, 20.0 g Mentha arvensis essential oil 15.0 g Linum usitatissimum Oil, 15.0 g Prunus armeniaca, 20.00 g Gaultheria procumbens Essential Oil, 20.00 g miconazole nitrate with 7.6 g sodium polyacrylate, 3.8 g poly oxy ethylene (20) sorbitan monolaurate and then mixing sufficiently to dissolve the sodium polyacrylate, followed by then adding 859 g cold water then rapidly mixing for 2 to 5 minutes for the stable single phase homogeneous cream to form. Mixing of the oil phase components with the sodium polyacrylate is limited to that required to dissolve the miconazole nitrate and sodium polyacrylate with the oil phase components. Following this step mixing of the water phase with the oil phase components containing the prior dissolved sodium polyacrylate and the poly oxy ethylene (20) sorbitan monolaurate, is required for a period of time (generally 2 to 5 minutes) to create the stable single phase homogeneous cream and to minimize volatilization of essential oils containing borneol, 1,8-cineole, 1-menthol and methyl salicylate. Methods of use of the composition of the topical cream given in this Example 17 include but are not meant to be limited to placing the cream composition in a roll-on bottle then placing the roll-on ball into the roll-on bottle container, placing the cream composition in a squeeze tube container, placing the cream composition in a hand pump bottle container and applying a therapeutic amount of the cream on the skin or wetting a patch with the cream and placing on the skin. The composition of a topical cream with borneol, 1,8-cineole, 1-menthol, methyl salicylate, miconazole nitrate and Omega-3 and Omega-9 fatty acids is shown in FIG. 17. In addition to treating dermatitis, pruritus, and fungal infections, the composition also has antibacterial properties.

### Example 18

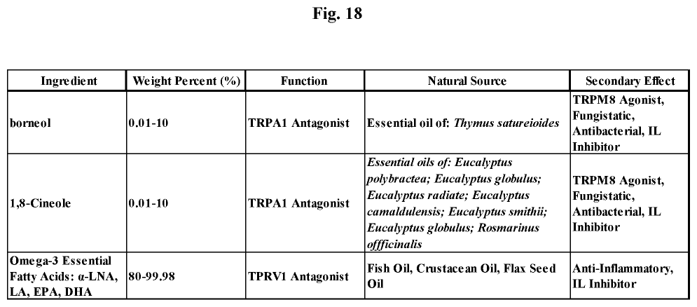

A composition and method of manufacture of an orally consumed nutritional supplement to treat dermatitis and pruritus comprising: borneol, 1,8-cineole, and Omega-3 and Omega-9 fatty acids for the composition presented in FIG. 18. The method of manufacture consists of borneol and 1,8-cineole together then mixing Omega-3 and Omega-9 fatty acids. Mixing required in minimal to create a stable single phase homogeneous oil. Methods of use and manufacturing of the composition of the nutritional supplement given in this Example 18 include, but are not meant to be limited to placing 0.5 g to 1.0 g of the mixture gelatin capsules, sealing the capsules with a microspray sealant, heat, or both, or alternatively using pressure. The nutritional supplement composition in this Example 18 can optionally be made with borneol alone or 1,8-cineole alone same total concentration range as 1,8-cineole and borneol presented in FIG. 18. In addition to treating dermatitis and pruritus, the composition also has fungistatic and antibacterial properties.

### Example 19

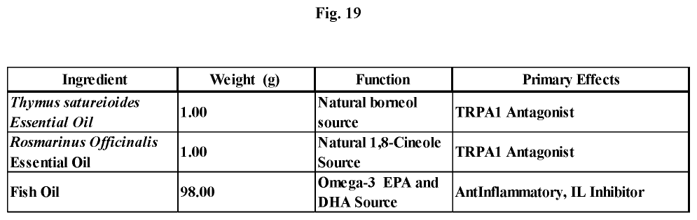

A preferred composition and method of manufacture of the nutritional supplement presented in this Example 19 to treat dermatitis and pruritus comprising: borneol, 1,8-cineole and Omega-3 and Omega-9 fatty acids is presented in FIG. 19. Manufacturing consists of mixing the oil phase components comprising 10.0 g Thymus satureioides 10.0 g Rosmarinus Officinalis Essential Oil, and 980.00 g Fish Oil and then mixing sufficiently to dissolve the ingredients until a single phase homogeneous oil results. Mixing intensity and duration is limited to that required to make a homogeneous oil and to minimize volatilization of essential oils containing borneol and 1,8-cineole. The composition can be placed in an amber glass bottle or various sizes, with and with a dispensing dropper or pump. Alternatively, the composition can be placed into 0.5 g to 1.0 g capsules either manually of automatically in capsules, which are preferably gelatin, then sealed. It is preferable the composition should be refrigerated soon after manufacturing. In addition to treating dermatitis and pruritus, the composition also has fungistatic and antibacterial properties. A person ordinarily skilled in the art would recognize that the fish oil could come from many sources and also could be from crustaceans, including krill. It is important to use the purest fish and crustacean oils to minimize the presence of heavy metals and other impurities, including but not limited to mercury and PCBs. A person ordinarily skilled in the art would also recognize that dependent on the precise composition of the borneol and 1,8-cineole in Thymus satureioides and Rosmarinus Officinalis essential oils used in the formulations that either a greater or smaller amount of the essential oils could be utilized. The nutritional supplement composition in this Example 19 can optionally be made with borneol alone or 1,8-cineole alone same total concentration range as 1,8-cineole and borneol presented in FIG. 19. A person ordinarily skilled in the art would also recognize that the selection of specific fish and krill oil and the method of concentrating EPA and DHA in the oils can vary and can be selected to result in specific EPA and DHA concentrations and to vary the EPA to DHA ratio. In addition to treating dermatitis and pruritus, the composition also has fungistatic and antibacterial properties.

### Example 20

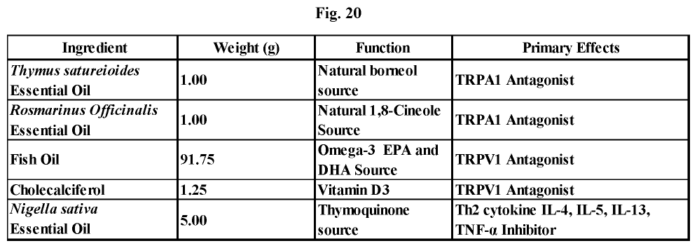

A preferred composition and method of manufacture of the nutritional supplement presented in this Example 20 to treat dermatitis and pruritus comprising: borneol, 1,8-cineole and Omega-3 and Omega-9 fatty acids, Vitamin D3, thymoquinone, optionally sodium hyaluronate, and optionally a cannabinoid compound is presented in FIG. 20. Manufacturing consists of mixing the oil phase components comprising 10.0 g Thymus satureioides 10.0 g Rosmarinus Officinalis Essential Oil. 50 g Nigella sativa essential oil and 917.50 g Fish Oil and then slowly mixing 12.5 g cholecalciferol and sufficiently to dissolve the cholecalciferol until a single phase homogeneous oil results. Mixing intensity and duration is limited to that required to dissolved the cholecalciferol and to make a homogeneous oil and to minimize volatilization of essential oils containing borneol and 1,8-cineole. The composition can be placed in an amber glass bottle or various sizes, with and with a dispensing dropper or pump. Alternatively, the composition can be placed into 0.5 g to 1.0 g capsules either manually or automatically in capsules, which are preferably gelatin, then sealed. It is preferable the composition should be refrigerated soon after manufacturing. In addition to treating dermatitis and pruritus, the composition also has fungistatic and antibacterial properties. A person ordinarily skilled in the art would recognize that the fish oil could come from many sources and also could be from crustaceans, including krill. It is important to use the purest fish and crustacean oils to minimize the presence of heavy metals and other impurities, including but not limited to mercury and PCBs. A person ordinarily skilled in the art would also recognize that dependent on the precise composition of the borneol, 1,8-cineole and thymoquinone in Thymus satureioides, Rosmarinus Officinalis and Nigella sativa essential oils used in the formulations that either a greater or smaller amount of the essential oils could be utilized. A person ordinarily skilled in the art would also recognize that the selection of specific fish and krill oil and the method of concentrating EPA and. DHA in the oils can vary and can be selected to result in specific EPA and DHA concentrations and to vary the EPA to DHA ratio. Optionally, sodium hyaluronate or cannabinoid compounds can be added to this composition.

While we have shown and described several embodiments in accordance with our disclosure, it is to be clearly understood that the same may be susceptible to numerous changes apparent to one skilled in the art. Therefore, we do not wish to be limited to the details shown and described but intend to show all changes and modifications that come within the scope of the appended claims.

What is claimed is:

1. A water-soluble topical pharmaceutical composition effective to treat dermatitis and associated pruritus comprising a natural plant extract TRPV1 antagonist, a natural plant extract TRPA1 antagonist, a natural plant extract TRPM8 agonist, a non-steroidal anti-inflammatory compound and a carrier which comprises; water, a thickening agent and a non-ionic surfactant, wherein the composition further comprises cannabinoid compound.

2-7. (canceled)

8. The composition of claim 1 further comprising an antibiotic.

9. The composition of claim 1 further comprising a fungistatic compound.

10. The composition of claim 1 in which the natural plant extract TRPV1 antagonist is selected from one or more fatty acids of the group comprising: an Omega-9 fatty acid oleic acid (OL); an Omega-3 fatty acid a-linolenic acid (ALA), an Omega-6 fatty acid linoleic acid (LA), and an Omega-3 fatty acid eicosapentaenoic acid (EPA).

11. (canceled)

12. The composition of claim 1 where the TRV1 antagonist is selected from one or more of the naturally occurring compounds, comprising: flax seed oil, apricot kernel oil, cannabidiol (CBD), cannabigerol (CBG), cannabidivarin (CBDV), and 9-THC Propyl Analogue (THC-V).

13. The composition of claim 1 in which the natural plant extract TRPA1 antagonist is selected from one or more naturally occurring compounds comprising: borneol, 1,8-cineole, camphor, (−)-fenchone, fenchyl alcohol, 2-methylisoborneol, norcamphor, cannabidiol (CBD), cannabichromene (CBC) and Cannabinol (CBN).

14. The composition of claim 13 where the source of the naturally occurring TRPA1 antagonists is from one or more of the following essential oils: Inula essential oil (Inula graveolens; Thyme borneol essential oil (Thymus satureioides); Gudun Oil (Dipterocarpus Turbinatus), Camphorwood essential oil, (Cinnamomum camphora. Chvar. Borneol), Camphor laurel essential oil (Cinnamomum camphora), Indonesian cinnamon essential oil (Cinnamomun burmanni), Sage essential oil (Salvia Officinalis), Eucalyptus essential oil (Eucalyptus polybractea, Eucalyptus camaldulensis, Eucalyptus smithii, Eucalyptus globulus, Eucalyptus radiate, Eucalyptus spp.), Rosemary essential oil (cineole type) (Rosmarinus Officinalis), Helichrysum gymnocephalum, Bay Laurel Essential Oil (Laurus nobilis), Cannabis essential oil (Cannabis sativa, Cannabis indica, and Cannabis ruderalis), Spanish Sage essential oil (Salvia lavandulifolia).

15-16. (canceled)

17. The composition of claim 1 in which the natural plant extract TRPM8 agonist is 1-menthol.

18. The composition of claim 17 in which the source of 1-menthol is selected from one or more essential oils selected from the group of: Mentha spp., including, but not limited to: Mentha piperita; and Mentha arvensis.

19-20. (cancelled)

21. The composition of claim 1, where the non-steroidal anti-inflammatory compound is methyl salicylate.

22. The composition of claim 21 in which the source of the methyl salicylate is an essential oil from Gaultheria procumbens (wintergreen).

23. The composition of claim 1 in which the cannabinoid compounds is selected from one or more of the group comprising cannabidiol (CBD), cannabigerol (CBG), cannabichromene (CBC), cannabidivarin (CBDV), and delta9-tetrahydrocannabinol (THC).

24. The composition of claim 1 where the cannabinoid compound is cannabidiol.

25-28. (canceled)

29. A non-ionic surfactant of claim 1 wherein the surfactant is selected from a group comprising:
polyoxyethylene (20) sorbitan monolaurate; polyoxyethylene (20) sorbitan monooleate;
polyoxyethylene (20) sorbitan monopalmitate; polyoxyethylene (20) sorbitan monostearate;
sorbitan trioctadecanoate; polyglyceryl-3 Stearate; polyglyceryl-3 palmitate; polyglyceryl-2 laurate; polyglyceryl-5 laurate; polyglyceryl-5 oleate; polyglyceryl-5 dioleate; and polyglyceryl-10 diisostearate.

30. A thickening agent composition of claim 27 wherein the thickening agent is selected from a carbomer, cacia, alginic acid, bentonite, carboxymethyl cellulose, ethylcellulose, hydroxyethyl cellulose, hydroxypropyl cellulose, magnesium aluminum silicate (Veegum), methylcellulose, poloxamers (Pluronics), polyvinyl alcohol, sodium alginate, tragacanth, guar gum, and xanthan gum.

31. The composition of claim 30 where the thickener is the carbomer sodium polyacrylate.

32-79. (canceled)

80. A multifunctional method to treat dermatitis and associated pruritus comprising a natural plant extract TRPV1 antagonist, a natural plant extract is a TRPA1 antagonist, a natural plant extract TRPM8 agonist, a non-steroidal anti-inflammatory compound and a carrier which comprises water, a thickening agent and a non-ionic surfactant, optionally sodium hyaluronate, optionally a cannabinoid compound, optionally one or more antihistamine compounds, optionally one or more topical steroids, optionally one or more antibiotic compounds and optionally one or more fungistatic compounds, comprising:
a) about 0.5 to about 10% by weight of a natural plant extract TRPV1 antagonist;
b) about 0.5 to about 10% by weight of a natural plant extract TRPA1 antagonist;
c) about 0.5 to about 10% by weight of a natural plant extract TRPM8 agonist;
d) about 0.01 to about 3.00% by weight of a natural plant extract non-steroidal anti-inflammatory compound;
e) a carrier comprising about 80 to about 97 water, about 0.05 to about 2.5% by weight of a non-ionic surfactant and about 0.1 to about 5.0% of a thickening agent;
f) optionally, about 0.001 to about 1.0% of one or more cannabinoid compounds;
g) optionally from about 0.01 to about 1.0% sodium hyaluronate
h) optionally from about 0.1 to about 2.0% of one or more antihistamine compounds
i) optionally from about 0.1 to about 2.5% of one or more topical steroid compounds;
j) optionally from 0.5 μg/mL to approximately 700 μg/mL of one or more antibiotic compounds; and
k) optionally from 0.1 to approximately 5.0% of one or more fungistatic compounds.

81. The method of claim 80 wherein:
component (a) comprises flax seed oil and apricot kernel oil;
component (b) comprises borneol;
component (c) comprises 1-menthol;
component (d) comprises methyl salicylate;
component (e) comprises a carrier comprising about 80 to about 97% water, about 0.05 to about 2.5% by weight of a non-ionic surfactant and about 0.1 to about 5.0% of a thickening agent;
component (f) comprises cannabidiol;
component (g) comprises sodium hyaluronate;
component (h) comprises diphenhydramine hydrochloride;
component (i) comprises hydrocortisone;
component (j) comprises a mixture of bacitracin, neomycin and polymyxin B sulfate;
component (k) comprises miconazole nitrate.

82-152. (canceled)

153. A composition of claim 1, further comprising:
a) about 0.5 to about 10% by weight of a natural plant extract TRPV1 antagonist;
b) about 0.5 to about 10% by weight of a natural plant extract TRPA1 antagonist;
c) about 0.5 to about 10% by weight of a natural plant extract TRPM8 agonist;
d) about 0.01 to about 3.00% by weight of a natural plant extract non-steroidal anti-inflammatory compound;
e) a carrier comprising about 80 to about 97% water, about 0.05 to about 2.5% by weight of a non-ionic surfactant and about 0.1 to about 5.0% of a thickening agent;
f) optionally, about 0.001 to about 1.0% of one or more cannabinoid compounds; and
g) optionally, about 0.01 to about 1.0% sodium hyaluronate.

154. A composition as in claim 153 wherein:
component (a) comprises flax seed oil and apricot kernel oil;
component (b) comprises borneol;
component (c) comprises 1-menthol;
component (d) comprises methyl salicylate;
component (e) comprises water, the non-ionic surfactant polyoxyethylene (20) sorbitan monolaurate and sodium polyacrylate
component (f) comprises cannabidiol; and
component (g) comprises sodium hyaluronate.
